SEQUENCE LISTING (56 sequences)

SEQ ID NO: 1 - protein, 118 aa, synthetic construct
EVQLVESGGGLVKPGGSLKLSCVASGFTLSNYAMSWVRQSPEKRLEWVAEVSSSGIYIYYSDTVTGRFSISRDNAKNTLYLEMSSLRSEDTAIYYCARDRYAYAMDYWGHGTSVIVSS

SEQ ID NO: 2 - protein, 5 aa, synthetic construct
NYAMS

SEQ ID NO: 3 - protein, 17 aa, synthetic construct
EVSSSGIYIYYSDTVTG

SEQ ID NO: 4 - protein, 9 aa, synthetic construct
DRYAYAMDY

SEQ ID NO: 5 - DNA, 356 bp, synthetic construct
  source 1..356: mol_type = other DNA
gaggtgcagctggtggaatccggcggaggcctggtgaaacctggcggctccctgaagctgtcctgcgtggcctccggcttcaccctgtccaactacgccatgtcctgggtgcgacagtcccccgagaagcggctggaatgggtggccgaggtctcctcctccggcatctacatctactactccgacaccgtgacaggccggttctccatctcccgggacaacgccaagaacaccctgtacctggaaatgtctagcctgcggagcgaggacaccgctatctactactgcgccagagacagatacgcctacgccatggactactggggccacggcacctccgtgatcgtgtcctctgc

SEQ ID NO: 6 - protein, 113 aa, synthetic construct
DIVMTQSPSSLAISVGQRVTLSCKSSQSLLNSYNQKNSLAWYQQKPGQSPKLLVYFASTRESGVPDRFIGSGSGTYFTLTITSVQAGDLADYFCQQHYSNPRTFGGGTRLEIK

SEQ ID NO: 7 - protein, 17 aa, synthetic construct
KSSQSLLNSYNQKNSLA

SEQ ID NO: 8 - protein, 7 aa, synthetic construct
FASTRES

SEQ ID NO: 9 - protein, 9 aa, synthetic construct
QQHYSNPRT

SEQ ID NO: 10 - DNA, 339 bp, synthetic construct
  source 1..339: mol_type = other DNA
gacatcgtgatgacccagtccccctccagcctggccatctccgtgggccagagagtgaccctgtcctgcaagtcctcccagtccctgctgaactcctacaaccagaagaactccctggcctggtatcagcagaagcccggccagtcccccaagctgctggtgtacttcgcctccacccgcgagtccggcgtgcccgacagattcatcggctccggctccggcacctacttcaccctgaccatcacctccgtgcaggccggcgacctggccgactacttctgccagcagcactactccaacccccggaccttcggcggaggcacccggctggaaatcaag

SEQ ID NO: 11 - protein, 120 aa, synthetic construct
QVQLKESGPGLVAPSQSLSITCTVSGFSLTRYDLSWIRQPPGKGLQWLGVIWTGGGTNYNSAFMSRLSIRKDNSKSQVFLKMNSLQTDDTAIYYCVRENYYNRRGYFDVWGAGTTVTVSS

SEQ ID NO: 12 - protein, 5 aa, synthetic construct
RYDLS

SEQ ID NO: 13 - protein, 16 aa, synthetic construct
VIWTGGGTNYNSAFMS

SEQ ID NO: 14 - protein, 12 aa, synthetic construct
ENYYNRRGYFDV

SEQ ID NO: 15 - DNA, 360 bp, synthetic construct
  source 1..360: mol_type = other DNA
caggtgcagctgaaagagtccggccctggcctggtggccccttcccagtccctgtccatcacctgtaccgtgtccggcttcagcctgaccagatacgacctgtcctggatcagacagccccctggcaagggcctgcagtggctgggagtgatctggaccggcggaggcaccaactacaactccgccttcatgtcccggctgtccatcagaaaggacaactccaagtcccaggtgttcctgaagatgaactccctgcagaccgacgacaccgccatctactactgcgtgcgcgagaactactacaacagacggggctacttcgacgtgtggggcgctggcaccaccgtgacagtgtcctct

SEQ ID NO: 16 - protein, 106 aa, synthetic construct
QIVLTQSPAIMSTSPGEKVTITCSASSSVSYMHWFQQKPGTSPKLWIYLTSNLASGVPARFSGSGSGTSYSLTINRMEAEDAATYYCQQRSGSPPTFGTGTKLELR

SEQ ID NO: 17 - protein, 10 aa, synthetic construct
SASSSVSYMH

SEQ ID NO: 18 - protein, 7 aa, synthetic construct
LTSNLAS

SEQ ID NO: 19 - protein, 9 aa, synthetic construct
QQRSGSPPT

SEQ ID NO: 20 - DNA, 318 bp, synthetic construct
  source 1..318: mol_type = other DNA
cagatcgtgctgacccagtcccccgccatcatgtccaccagccctggcgagaaagtgacaatcacctgttccgccagctcctccgtgtcctacatgcactggttccagcagaagcccggcacctcccccaagctgtggatctacctgacctccaacctggcctccggcgtgcccgccagattctctggctccggctccggcaccagctactccctgaccatcaaccggatggaagccgaggacgccgccacctactactgccagcagagatccggctccccccctaccttcggcaccggcaccaaactggaactgcgg

SEQ ID NO: 21 - protein, 118 aa, synthetic construct
EVQLVESGGGVVQPGRSLRLSCAASGFTLSNYAMSWVRQAPGKGLEWVAEVSSSGIYIYYSDTVTGRFTISRDNSKNTLYLQMNSLRAEDTAVYYCARDRYAYAMDYWGQGTLVTVSS

SEQ ID NO: 22 - DNA, 354 bp, synthetic construct
  source 1..354: mol_type = other DNA
gaagtgcagctggtggaatccggcggaggcgtggtgcagcctggcagatccctgagactgtcctgcgccgcctccggcttcaccctgtccaactacgccatgtcctgggtccgacaggcccctggcaagggcctggaatgggtggccgaggtgtcctcctccggcatctacatctactactccgacaccgtgacaggccggttcaccatctcccgggacaactccaagaacaccctgtacctgcagatgaactccctgcgggccgaggacaccgccgtgtactactgcgccagagacagatacgcctacgccatggactactggggccagggcaccctggtcaccgtgtcctct

SEQ ID NO: 23 - protein, 113 aa, synthetic construct
DIVMTQSPDSLAVSLGERATINCKSSQSLLNSYNQKNSLAWYQQKPGQPPKLLIYFASTRESGVPDRFSGSGSGTDFTLTISSLQAEDVAVYYCQQHYSNPRTFGGGTKLEIK

SEQ ID NO: 24 - DNA, 339 bp, synthetic construct
  source 1..339: mol_type = other DNA
gacatcgtgatgacccagtcccccgactccctggccgtgtctctgggcgagcgggccaccatcaactgcaagtcctcccagtccctgctgaactcctacaaccagaagaacagcctggcgtggtatcagcagaagcccggccagccccccaagctgctgatctacttcgcctccacccgcgagtccggcgtgcccgatagattctccggctccggcagcggcaccgactttaccctgaccatcagctccctgcaggccgaggacgtggccgtgtactactgccagcagcactactccaacccccggaccttcggcggaggcaccaagctggaaatcaaa

SEQ ID NO: 25 - protein, 120 aa, synthetic construct
EVQLVESGGGLVQPGGSLRLSCAVSGFSLTRYDLSWVRQAPGKGLEWLGVIWTGGGTNYNSAFMSRLTISRDNSKNTLYLQMNSLRAEDTAVYYCVRENYYNRRGYFDVWGQGTTVTVSS

SEQ ID NO: 26 - DNA, 360 bp, synthetic construct
  source 1..360: mol_type = other DNA
gaagtgcagctggtcgagtctggcggagggctcgtgcagcctggcggatctctgagactgtcctgtgccgtgtccggcttcagcctgaccagatacgacctgtcctgggtccgacaggctcctggcaagggcctggaatggctgggcgtgatctggaccggcggaggcaccaactacaactccgccttcatgtcccggctgaccatctcccgggacaactccaagaacaccctgtacctgcagatgaactccctgcgggccgaggacaccgccgtgtactactgcgtgcgcgagaactactacaacagacggggctacttcgacgtgtggggccagggcaccactgtcaccgtgtcctcc

SEQ ID NO: 27 - protein, 120 aa, synthetic construct
QVQLQESDPGLVKPSETLSLTCTVSGFSISRYDLSWIRQPPGKGLEWIGVIWTGGGTNYNSAFMSRLTISKDTSKNQVSLKLSSVTAADTAVYYCARENYYNRRGYFDVWGQGTMVTVSS

SEQ ID NO: 28 - DNA, 541 bp, synthetic construct
  source 1..541: mol_type = other DNA
ggcgaattggcggaaggccgtcaaggcctaggcgcgccatgagctcaagcttgccgccaccatggagtggtccggcatcttcctgtttctggtggctaccgccaccgacgtgcactctcaagtgcagctgcaagaatccgaccccggcctcgtgaaaccctccgagacactgtccctgacctgcaccgtgtccggcttctccatcagcagatacgacctgtcctggatcagacagccccctggcaagggcctggaatggatcggcgtgatctggaccggcggaggcaccaactacaactccgccttcatgtcccggctgaccatctccaaggacacctccaagaaccaggtgtccctgaagctgtcctccgtgaccgccgctgacaccgccgtgtactactgcgccagagagaactactacaacagacggggctacttcgacgtgtggggccagggcaccatggtgacagtgtcctccgcctccaccaagggcccggtacctcttaattaactggcctcatgggccttccgctcactgccc

SEQ ID NO: 29 - protein, 106 aa, synthetic construct
EIVLTQSPATLSLSPGERATLSCSASSSVSYMHWFQQKPGQAPRLLIYLTSNLASGIPARFSGSGSGTDFTLTISSLEPEDFAVYYCQQRSGSPPTFGPGTKLDIK

SEQ ID NO: 30 - DNA, 318 bp, synthetic construct
  source 1..318: mol_type = other DNA
gagatcgtgctgacccagtcccctgccaccctgtctctgagccctggcgagagagccaccctgagctgctccgcctcctcctccgtgtcctacatgcactggttccagcagaagcccggccaggcccctcggctgctgatctacctgacctccaacctggcctccggcatccctgcccggttctctggctccggctccggcaccgactttaccctgaccatctccagcctggaacccgaggacttcgccgtgtactactgccagcagcggagcggctccccccctacatttggccctggcaccaagctggacatcaaa

SEQ ID NO: 31 - protein, 113 aa, synthetic construct
DIVMTQSPDSLAVSLGERATINCKSSQSLLNSYNQKNSLAWYQQKPGQPPKLLVYFASTRESGVPDRFSGSGSGTDFTLTISSLQAEDVAVYFCQQHYSNPRTFGGGTKLEIK

SEQ ID NO: 32 - DNA, 339 bp, synthetic construct
  source 1..339: mol_type = other DNA
gacatcgtgatgacccagtcccccgactccctggccgtgtctctgggcgagcgggccaccatcaactgcaagtcctcccagtccctgctgaactcctacaaccagaagaacagcctggcgtggtatcagcagaagcccggccagccccccaagctgctggtgtacttcgcctccacccgcgagtctggcgtgcccgacagattctccggctccggcagcggcaccgactttaccctgaccatcagctccctgcaggccgaggacgtggccgtgtacttctgccagcagcactactccaacccccggaccttcggcggaggcaccaagctggaaatcaaa

SEQ ID NO: 33 - protein, 118 aa, synthetic construct
EVQLVESGGGVVQPGRSLRLSCAASGFTLSNYAMSWVRQAPGKGLEWVAEVSSSGIYIYYSDTVTGRFSISRDNSKNTLYLQMNSLRAEDTAVYYCARDRYAYAMDYWGQGTLVIVSS

SEQ ID NO: 34 - DNA, 354 bp, synthetic construct
  source 1..354: mol_type = other DNA
gaagtgcagctggtggaatccggcggaggcgtggtgcagcctggcagatccctgagactgtcctgcgccgcctccggcttcaccctgtccaactacgccatgtcctgggtgcgacaggcccctggcaagggactggaatgggtggccgaggtgtcctcctccggcatctacatctactactccgacaccgtgaccggccggttctccatctcccgggacaactccaagaacaccctgtacctgcagatgaattccctgcgggccgaggacaccgccgtgtactactgcgccagagacagatacgcctacgccatggactactggggccagggcaccctggtgatcgtgtcctcc

SEQ ID NO: 35 - protein, 113 aa, synthetic construct
DIVMTQSPDSLAVSLGERATINCKSSQSLLNSYNQKNSLAWYQQKPGQPPKLLVYFASTRESGVPDRFIGSGSGTYFTLTISSLQAEDVAVYFCQQHYSNPRTFGGGTKLEIK

SEQ ID NO: 36 - DNA, 339 bp, synthetic construct
  source 1..339: mol_type = other DNA
gacatcgtgatgacccagtcccccgactccctggccgtgtctctgggcgagcgggccaccatcaactgcaagtcctcccagtccctgctgaactcctacaaccagaagaacagcctggcctggtatcaacagaagcccggccagccccccaagctgctggtgtacttcgcctccacccgcgagtccggcgtgcccgacagattcatcggctccggctccggcacctacttcaccctgaccatcagctccctgcaggccgaggacgtggccgtgtacttctgccagcagcactactccaacccccggaccttcggcggaggcaccaagctggaaatcaaa

SEQ ID NO: 37 - protein, 120 aa, synthetic construct
EVQLVESGGGLVQPGGSLRLSCAVSGFSLTRYDLSWVRQAPGKGLEWLGVIWTGGGTNYNSAFMSRFSISKDNSKNTVFLQMNSLRAEDTAVYYCARENYYNRRGYFDVWGQGTTVTVSS

SEQ ID NO: 38 - DNA, 360 bp, synthetic construct
  source 1..360: mol_type = other DNA
gaagtgcagctggtggaatccggcggaggactggtgcaacctggcggctccctgagactgtcttgcgccgtgtccggcttcagcctgaccagatacgacctgtcctgggtgcgacaggcccctggcaagggcctggaatggctgggcgtgatctggaccggaggaggcaccaactacaactccgccttcatgtcccggttctccatctccaaggacaactccaagaacaccgtgtttctgcagatgaactccctgcgggctgaggacaccgccgtgtactactgcgccagagagaactactacaacagacggggctacttcgacgtgtggggccagggcaccaccgtgaccgtgtcctct

SEQ ID NO: 39 - protein, 106 aa, synthetic construct
QIVLTQSPATLSLSPGERATLSCSASSSVSYMHWFQQKPGQAPRLLIYLTSNLASGVPARFSGSGSGTDFTLTISSLEPEDFAVYYCQQRSGSPPTFGPGTKLDIK

SEQ ID NO: 40 - DNA, 318 bp, synthetic construct
  source 1..318: mol_type = other DNA
cagatcgtgctgacccagtcccctgccaccctgtctctgagccctggcgagagagccaccctgagctgctccgcctcctcctccgtgtcctacatgcactggttccagcagaagcccggccaggcccctcggctgctgatctacctgacctccaacctggcctccggcgtgcccgccagattctctggctccggctccggcaccgacttcaccctgaccatctccagcctggaacccgaggacttcgccgtgtactactgccagcagcggagcggctccccccctacctttggccctggcaccaagctggacatcaaa

SEQ ID NO: 41 - protein, 113 aa, synthetic construct
DIVMTQSPSSLAISVGQRVTLSCKSSQSLLNSYNQKNSLAWYQQKPGQPPKLLIYFASTRESGVPDRFSGSGSGTDFTLTISSLQAEDVAVYYCQQHYSNPRTFGGGTKLEIK

SEQ ID NO: 42 - DNA, 339 bp, synthetic construct
  source 1..339: mol_type = other DNA
gacatcgtgatgacccagtccccctccagcctggccatctccgtgggccagagagtgaccctgtcctgcaagtcctcccagtccctgctgaactcctacaaccagaagaacagcctggcgtggtatcagcagaagcccggccagccccccaagctgctgatctacttcgcctccacccgcgagtccggcgtgcccgatagattctccggctccggcagcggcaccgactttaccctgaccatcagctccctgcaggccgaggacgtggccgtgtactactgccagcagcactactccaacccccggaccttcggcggaggcaccaagctggaaatcaaa

SEQ ID NO: 43 - protein, 113 aa, synthetic construct
DIVMTQSPDSLAVSLGERATINCKSSQSLLNSYNQKNSLAWYQQKPGQSPKLLVYFASTRESGVPDRFSGSGSGTDFTLTISSLQAEDVAVYYCQQHYSNPRTFGGGTKLEIK

SEQ ID NO: 44 - DNA, 339 bp, synthetic construct
  source 1..339: mol_type = other DNA
gacatcgtgatgacccagtcccccgactccctggccgtgtctctgggcgagcgggccaccatcaactgcaagtcctcccagtccctgctgaactcctacaaccagaagaacagcctggcgtggtatcagcagaagcccggccagtcccccaagctgctggtgtacttcgcctccacccgcgagtccggcgtgcccgatagattctccggctccggcagcggcaccgactttaccctgaccatcagctccctgcaggccgaggacgtggccgtgtactactgccagcagcactactccaacccccggaccttcggcggaggcaccaagctggaaatcaaa

SEQ ID NO: 45 - protein, 113 aa, synthetic construct
DIVMTQSPDSLAVSLGERATINCKSSQSLLNSYNQKNSLAWYQQKPGQPPKLLIYFASTRESGVPDRFIGSGSGTYFTLTITSLQAEDVAVYYCQQHYSNPRTFGGGTKLEIK

SEQ ID NO: 46 - DNA, 339 bp, synthetic construct
  source 1..339: mol_type = other DNA
gacatcgtgatgacccagtcccccgactccctggccgtgtctctgggcgagcgggccaccatcaactgcaagtcctcccagtccctgctgaactcctacaaccagaagaacagcctggcgtggtatcagcagaagcccggccagccccccaagctgctgatctacttcgcctccacccgcgagtccggcgtgcccgacagattcatcggctccggctccggcacctacttcaccctgaccatcacctccctgcaggccgaggacgtggccgtgtactactgccagcagcactactccaacccccggaccttcggcggaggcaccaagctggaaatcaaa

SEQ ID NO: 47 - protein, 113 aa, synthetic construct
DIVMTQSPDSLAVSLGERATINCKSSQSLLNSYNQKNSLAWYQQKPGQPPKLLIYFASTRESGVPDRFSGSGSGTDFTLTISSVQAGDLADYFCQQHYSNPRTFGGGTKLEIK

SEQ ID NO: 48 - DNA, 339 bp, synthetic construct
  source 1..339: mol_type = other DNA
gacatcgtgatgacccagtcccccgactccctggccgtgtctctgggcgagcgggccaccatcaactgcaagtcctcccagtccctgctgaactcctacaaccagaagaacagcctggcgtggtatcagcagaagcccggccagccccccaagctgctgatctacttcgcctccacccgcgagtccggcgtgcccgatagattctccggctccggcagcggcaccgactttaccctgaccatcagctccgtgcaggccggcgacctggccgactacttctgccagcagcactactccaacccccggaccttcggcggaggcaccaagctggaaatcaaa

SEQ ID NO: 49 - protein, 330 aa, Homo sapiens
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - DNA, 990 bp, Homo sapiens
  source 1..990: mol_type = genomic DNA
gcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagagagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggaggagatgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctatagcaagctcaccgtggacaagagcaggtggcagcaggggaacgtcttctcatgctccgtgatgcatgaggctctgcacaaccactacacgcagaagagcctctccctgtccccgggtaaa

SEQ ID NO: 51 - protein, 107 aa, Homo sapiens [sequence omitted]

SEQ ID NO: 52 - DNA, 321 bp, Homo sapiens
  source 1..321: mol_type = genomic DNA
cgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 53 - protein, 360 aa, Homo sapiens
MLSTSRSRFIRNTNESGEEVTTFFDYDYGAPCHKFDVKQIGAQLLPPLYSLVFIFGFVGNMLVVLILINCKKLKCLTDIYLLNLAISDLLFLITLPLWAHSAANEWVFGNAMCKLFTGLYHIGYFGGIFFIILLTIDRYLAIVHAVFALKARTVTFGVVTSVITWLVAVFASVPGIIFTKCQKEDSVYVCGPYFPRGWNNFHTIMRNILGLVLPLLIMVICYSGILKTLLRCRNEKKRHRAVRVIFTIMIVYFLFWTPYNIVILLNTFQEFFGLSNCESTSQLDQATQVTETLGMTHCCINPIIYAFVGEKFRRYLSVFFRKHITKRFCKQCPVFYRETVDGVTSTNTPSTGEQEVSAGL

SEQ ID NO: 54 - protein, 359 aa, Callithrix jacchus
MLSTSHSRFIRNTESGEEVTTIFDYDYGAPCHKFDVKQIGAQLLPPLYSLVFIFGFVGNMLVVLILINCKKLKSLTDIYLLNLAVSDLLFLITLPLWAHSAANEWVFGNAVCKLFTGLYHIGYFGGIFFIILLTIDRYLAIVHAVFALKARTVTFGVVTSVITWFVAVFASVPGIIFTKSQKEDSVYVCGPYFPRGWNHFHTIMRNLLGLVLPLLVMIICYSGILKTLLRCRNEKKRHRAVRLIFTIMIVYFLFWTPYNIVVLLNTFQEFFGLSNCESTSQLDQATQVTETLGMTHCCINPIIYAFVGEKFRRYLSVFFRKHIAKRFCKQCPVFYRETVDGVTSTNTPSTGEQEVSAGL

SEQ ID NO: 55 - protein, 360 aa, Macaca mulatta
MLSTSRSRFIRNTNGSGEEVTTFFDYDYGAPCHKFDVKQIGAQLLPPLYSLVFIFGFVGNMLVVLILINCKKLKSLTDIYLLNLAISDLLFLITLPLWAHSAANEWVFGNAMCKLFTGLYHIGYLGGIFFIILLTIDRYLAIVHAVFALKARTVTFGVVTSVITWLVAVFASVPGIIFTKCQEEDSVYICGPYFPRGWNNFHTIMRNILGLVLPLLIMVICYSGILKTLLRCRNEKKRHRAVRLIFTIMIVYFLFWTPYNIVILLNTFQEFFGLSNCESTRQLDQATQVTETLGMTHCCINPIIYAFVGEKFRRYLSMFFRKYITKRFCKQCPVFYRETVDGVTSTNTPSTAEQEVSVGL

SEQ ID NO: 56 - protein, 99 aa, Homo sapiens
MKVSAALLCLLLIAATFIPQGLAQPDAINAPVTCCYNFTNRKISVQRLASYRRITSSKCPKEAVIFKTIVAKEICADPKQKWVQDSMDHLDKQTQTPKT

(19) United States
(12) Patent Application Publication
(10) Pub. No.: US 2026/0193365 A1
(43) Pub. Date: Jul. 9, 2026

(54) ANTIBODIES TARGETING CCR2

(71) Applicant: Granite Bio AG, Basel (CH)

(72) Inventors: Matthias MACK, Regensburg (DE); Eilish CULLEN, Clacton On Sea Essex (GB); David MATTHEWS, Hitchin (GB)

(21) Appl. No.: 19/402,360

(22) Filed: Nov. 26, 2025

Related U.S. Application Data
(63) Continuation of application No. PCT/EP2024/064489, filed on May 27, 2024.

(30) Foreign Application Priority Data
May 29, 2023 (EP) 23175902.8

(51) Int. Cl. C07K 16/28

(52) U.S. Cl. CPC C07K 16/2866; C07K 2317/24

(57) ABSTRACT

The present disclosure relates to antibodies and antibody fragment that are specific for CCR2. Almost all attempts to humanize the originator antibody failed, due to loss of reactivity with marmoset CCR2, an essential species for further pre-clinical and clinical development. The antibodies are useful for the treatment of inflammatory diseases, autoimmune diseases, hematologic malignancies and potentially other illnesses.

### RELATED APPLICATIONS

This application is a U.S. continuation application of PCT/EP2024/064489, filed May 27, 2024, which claims priority to European Application No. 23175902.8, filed May 29, 2023, the entire disclosures of which are hereby incorporated by reference herein.

### REFERENCE TO SEQUENCE LISTING

This application contains a sequence listing which has been submitted electronically in XML format and is hereby incorporated by reference in its entirety (said XML copy, created May 7, 2024, is named “GRBI-001_01US_360810-2001_SeqList_ST26.xml” and is 60,545 bytes in size).

### FIELD OF THE INVENTION

The present disclosure relates to antibodies and antibody fragment that are specific for CCR2. Almost all attempts to humanize the originator antibody failed, for reasons such as the loss of cross-reactivity with marmoset CCR2, an essential species for further pre-clinical and clinical development. The antibodies generated in the present invention are useful for the treatment of inflammatory diseases, autoimmune diseases, hematologic malignancies and potentially other illnesses.

### BACKGROUND OF THE INVENTION

Monocytes migrate to sites of inflammation under the influence of chemokines and cytokines. One such chemokine is MCP-1 that binds to the chemokine receptor CCR2, which causes monocyte to egress from the bone marrow. MCP-1, which is secreted by cells during inflammatory diseases, autoimmune diseases, hematologic malignancies and other illnesses.

Several molecules targeting the MCP-1/CCR2 pathway were or are still tested and some showed promising results, but none was approved so far. Most of the drugs developed are small organic molecules. For example cenicrivirc, a small molecule developed by Takeda and Tobira Therapeutics, is currently in Phase 3 clinical testing. Cenicrivirc is however not specific for CCR2, but also inhibits CCR5.

STI-B0201 is an antibody generated by Sorrento, but no clinical development is reported. Anti-CCR2 antibodies of Sorrento are disclosed in WO2013/192596. Unlike the antibodies of the present disclosure, the antibodies of WO2013/192596 do cross react with mouse CCR2. Antibodies of Anti-CCR2 antibodies are also disclosed in WO2007/115713 and U.S. Pat. No. 9,068,002. These are however murine antibodies which, likewise, never were developed. MLNM1202 (plozalizumab) is another anti-CCR2 antibody, but development was discontinued by Millennium. Antibodies of Takeda/Millennium are disclosed in WO2016/079276. Antibodies of Amgen are disclosed in US2011/0274696. The antibodies of US2011/0274696 show reactivity to cynomolgus CCR2, whereas the antibodies of the present disclosure do not cross react with cynomolgus CCR2. The antibodies of the present disclosure however do cross react with marmoset CCR2. Antibodies against CCR2 of the University of Regensburg/Yeda/Tel Aviv Medical Center are disclosed in U.S. Pat. No. 9,068,002. Antibodies against CCR2 of Pfizer are disclosed in WO2010021697. Yet again, no development is reported.

Hence, whilst there is a need and a great interest in developing specific anti-CCR2 targeting moieties, there is currently no anti-CCR2 antibody in advanced development. The present invention discloses novel antibodies that are highly specific for CCR2. Humanization of the originator antibody was extremely difficult, since essentially all humanized derivatives lost of reactivity to marmoset CCR2, an essential species for further pre-clinical and clinical development.

### SUMMARY OF THE INVENTION

The present disclosure relates to a humanized antibody or antibody fragment specific for human CCR2, wherein said antibody or antibody fragment comprises the HCDR1 region of SEQ ID NO: 2, the HCDR2 region of SEQ ID NO: 3, the HCDR3 region of SEQ ID NO: 4, the LCDR1 region of SEQ ID NO: 7, the LCDR2 region of SEQ ID NO: 8 and the LCDR3 region of SEQ ID NO: 9.

The present disclosure also relates to a humanized antibody or antibody fragment, wherein said antibody or antibody fragment comprises the VH of SEQ ID NO: 21 and the VL of SEQ ID NO 43.

In certain embodiments, the humanized antibody is of the human IgG1 class.

In certain embodiments, the humanized antibody or antibody fragment binds to the first extracellular loop of human CCR2 (SEQ ID No. 53) and the second extracellular loop of human CCR2 (SEQ ID No. 54).

In certain embodiments, the humanized antibody or antibody fragment according is cross-reactive with marmoset CCR2.

In certain embodiments, the humanized antibody or antibody fragment according binds to the first extracellular loop of marmoset CCR2 (SEQ ID No. 53) and the second extracellular loop of marmoset CCR2 (SEQ ID No. 55).

In certain embodiments, the humanized antibody or antibody fragment does not bind to rhesus CCR2.

The present disclosure also relates to the humanized antibody or antibody fragments disclosed herein for use in medicine, preferably wherein said treatment is the treatment of an inflammatory disease, an autoimmune diseases or a hematologic malignancy.

The present disclosure also relates to a nucleic acid composition comprising a nucleic acid sequence or a plurality of nucleic acid sequences encoding the isolated antibody or antibody fragments disclosed herein.

The present disclosure also relates to a vector comprising aforementioned nucleic acid compositions.

The present disclosure also relates to a host cell comprising aforementioned vector or nucleic acid compositions.

The present disclosure also relates to a pharmaceutical composition comprising the humanized antibody or antibody fragments disclosed herein and a pharmaceutically acceptable carrier or excipient.

### FIGURE LEGENDS

FIG. 1 shows the specific binding of the antibodies DOC2 chim. and DOC3 chim., as well as the control antibody AMBA, to human monocytes.

FIG. 2 compares the specific binding of the antibodies DOC2 chim. and DOC2-reshaped, as well as the control antibody AMBA, to human monocytes.

FIG. 3 compares the specific binding of the antibodies DOC3-chim., DOC3-RH03XRKA and DOC3-RHLXRKA, as well as the control antibody AMBA, to human monocytes.

FIG. 4 compares the specific binding of the antibodies DOC2 chim, and DOC2-reshaped, as well as the control antibody AMBA, to primary marmoset monocytes.

FIG. 5 compares the specific binding of the antibodies DOC3-chim., DOC3-RH03XRKA and DOC3-RHLXRKA, as well as the control antibody AMBA, to primary marmoset monocytes.

FIG. 6 compares the specific binding of the additionally humanized DOC2 antibodies to primary human monocytes.

FIG. 7 compares the specific binding of the additionally humanized DOC2 antibodies to primary marmoset monocytes.

FIG. 8 compares the specific binding of the additionally humanized DOC3 antibodies to primary human monocytes.

FIG. 9 compares the specific binding of the additionally humanized DOC3 antibodies to primary marmoset monocytes.

FIG. 10 compares the specific binding of antibodies DOC2 RHAXRKA FR1, DOC2 RHAXRKA FR2, DOC2 RHAXRKA FR3a, DOC2 RHAXRKA FR3b1 and DOC2 Chim. to primary human monocytes.

FIG. 11 compares the specific binding of antibodies DOC2 RHAXRKA FR1, DOC2 RHAXRKA FR2, DOC2 RHAXRKA FR3a, DOC2 RHAXRKA FR3b1 and DOC2 Chim. to primary marmoset monocytes.

FIG. 12 shows that DOC2-RHAXRKA FR2, but not rituximab or the human IgG1 isotype control antibody significantly depletes classical CD14+ monocytes. hDOC2=DOC2-RHAXRKA FR2; hIgG1 Iso=human IgG1 isotype control antibody.

FIG. 13 shows that rituximab, but not DOC2-RHAXRKA FR2 or the human IgG1 isotype control antibody deplete B cells. hDOC2=DOC2-RHAXRKA FR2; hIgG1 Iso=human IgG1 isotype control antibody.

FIG. 14 shows that none of the antibodies tested depletes T cells. hDOC2=DOC2-RHAXRKA FR2; hIgG1 Iso=human IgG1 isotype control antibody.

FIG. 15 shows that DOC2-RHAXRKA FR2 upregulates CD11b median fluorescence intensity on NK cells. hDOC2=DOC2-RHAXRKA FR2; hIgG1 Iso=human IgG1 isotype control antibody.

FIG. 16 shows that DOC2-RHAXRKA FR2 downregulates CD16 median fluorescence intensity on NK cells. hDOC2=DOC2-RHAXRKA FR2; hIgG1 Iso=human IgG1 isotype control antibody.

### Definitions

The disclosure pertains to antibodies, which specifically bind to CCR2.

The term “CCR2” refers to a protein known as C-C Motif Chemokine Receptor 2, also known as CD192 or MCP-1 receptor. Human CCR2 exists in two main isoforms, CCR2a and CCR2b, which are produced by alternative splicing. CCR2a and CCR2b only differ at the cytosolic, C-terminal part. The extracellular part which is accessible to therapeutic antibodies and antibody fragments is identical in both isoforms.

Human CCR2b has the amino acid sequence of
(UniProtKB/Swiss-Prot: P41597):
(SEQ ID NO: 53)
MLSTSRSRFIRNTNESGEEVTTFFDYDYGAPCHKFDVKQIGAQLL
PPLYSLVFIFGFVGNMLVVLILINCKKLKCLTDIYLLNLAISDLL
FLITLPLWAHSAANEWVFGNAMCKLFTGLYHIGYFGGIFFIILLT
IDRYLAIVHAVFALKARTVTFGVVTSVITWLVAVFASVPGIIFTK
CQKEDSVYVCGPYFPRGWNNFHTIMRNILGLVLPLLIMVICYSGI
LKILLRCRNEKKRHRAVRVIFTIMIVYFLFWTPYNIVILLNTFQE
FFGLSNCESTSQLDQATQVTETLGMTHCCINPIIYAFVGEKFRRY
LSVFFRKHITKRFCKQCPVFYRETVDGVTSINTPSTGEQEVSAGL
Marmoset monkey (Callithrix jacchus) CCR2b
has the amino acid sequence of
(SEQ ID NO: 54)
MLSTSHSRFIRNTESGEEVTTIFDYDYGAPCHKEDVKQIGAQLLP
PLYSLVFIFGFVGNMLVVLILINCKKLKSLTDIYLLNLAVSDLLF
LITLPLWAHSAANEWVFGNAVCKLFTGLYHIGYFGGIFFIILLTI
DRYLAIVHAVFALKARTVTFGVVTSVITWFVAVFASVPGIIFTKS
QKEDSVYVCGPYFPRGWNHFHTIMRNLLGLVLPLLVMIICYSGIL
KILLRCRNEKKRHRAVRLIFTIMIVYFLFWTPYNIVVLLNTFQEF
FGLSNCESTSQLDQATQVTETLGMTHCCINPIIYAFVGEKFRRYL
SVFFRKHIAKRFCKQCPVFYRETVDGVTSTNTPSTGEQEVSAGL
Rhesus monkey (Macaca mulatta) CCR2b has
the amino acid sequence of
(SEQ ID NO: 55)
MLSTSRSRFIRNINGSGEEVTTFFDYDYGAPCHKFDVKQIGAQLL
PPLYSLVFIFGFVGNMLVVLILINCKKLKSLIDIYLLNLAISDLL
FLITLPLWAHSAANEWVFGNAMCKLFTGLYHIGYLGGIFFIILLT
IDRYLAIVHAVFALKARTVTFGVVTSVITWLVAVFASVPGIIFTK
CQEEDSVYICGPYFPRGWNNFHTIMRNILGLVLPLLIMVICYSGI
LKILLRCRNEKKRHRAVRLIFTIMIVYFLFWTPYNIVILLNTFQE
FFGLSNCESTRQLDQATQVTETLGMTHCCINPIIYAFVGEKFRRY
LSMFFRKYITKRFCKQCPVFYRETVDGVTSINTPSTAEQEVSVGL

CCR2 is the key functional receptor for MCP-1. Its binding to MCP-1 mediates chemotaxis and migration of monocytes and macrophages.

The term “MCP-1” refers to a protein known as Monocyte chemotactic Protein 1. It is also known as CCL2 (C-C Motif Chemokine Ligand 2). Human MCP-1 has the amino acid sequence of (UniProtKB/Swiss-Prot: P13500):

(SEQ ID NO: 56)
MKVSAALLCLLLIAATFIPQGLAQPDAINAPVTCCYNFTNRKISV
QRLASYRRITSSKCPKEAVIFKTIVAKEICADPKQKWVQDSMDHL
DKQTQTPKT

The term “antibody” as used herein refers to a protein comprising at least two heavy (H) chains and two light (L) chains inter-connected by disulfide bonds, which interacts with an antigen. Each heavy chain is comprised of a heavy chain variable region (abbreviated herein as “VH”) and a heavy chain constant region. The heavy chain constant region is comprised of three domains, CH1, CH2 and CH3. Each light chain is comprised of a light chain variable region (abbreviated herein as “VL”) and a light chain constant region. The light chain constant region is comprised of one domain, CL. The VH and VL regions can be further subdivided into regions of hypervariability, termed complementarity determining regions (CDR), interspersed with regions that are more conserved, termed framework regions (FR). Each VH and VL is composed of three CDRs and four FR's arranged from amino-terminus to carboxy-terminus in the following order: FR1, CDR1, FR2, CDR2, FR3, CDR3, and FR4. The variable regions of the heavy and light chains contain a binding domain that interacts with an antigen. The constant regions of the antibodies may mediate the binding of the immunoglobulin to host tissues or factors, including various cells of the immune system (e.g., effector cells) and the first component (C1q) of the classical complement system. The term “antibody” includes for example, monoclonal antibodies, human antibodies, humanized antibodies, camelised antibodies and chimeric antibodies. The antibodies can be of any isotype (e.g., IgG, IgE, IgM, IgD, IgA and IgY), class (e.g., Igd, IgG2, IgG3, IgG4, IgA1 and IgA2) or subclass. Both the light and heavy chains are divided into regions of structural and functional homology.

The term “antibody fragment”, as used herein, refers to one or more portions of an antibody that retain the ability to specifically interact with (e.g., by binding, steric hindrance, stabilizing spatial distribution) an antigen. Examples of binding fragments include, but are not limited to, a Fab fragment, a monovalent fragment consisting of the VL, VH, CL and CH1 domains; a F (ab) 2 fragment, a bivalent fragment comprising two Fab fragments linked by a disulfide bridge at the hinge region; a Fd fragment consisting of the VH and CH1 domains; a Fv fragment consisting of the VL and VH domains of a single arm of an antibody; a dAb fragment (Ward et al., (1989) Nature 341:544-546), which consists of a VH domain; and an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al., (1988) Science 242:423-426; and Huston et al., (1988) Proc. Natl. Acad. Sci. 85:5879-5883). Such single chain antibodies are also intended to be encompassed within the term “antibody fragment”. These antibody fragments are obtained using conventional techniques known to those of skill in the art, and the fragments are screened for utility in the same manner as are intact antibodies. Antibody fragments can also be incorporated into single domain antibodies, maxibodies, minibodies, intrabodies, diabodies, triabodies, tetrabodies, v-NAR and bis-scFv (see, e.g., Hollinger and Hudson, (2005) Nature Biotechnology 23:1 126-1 136). Antibody fragments can be grafted into scaffolds based on polypeptides such as Fibronectin type III (Fn3) (see U.S. Pat. No. 6,703,199, which describes fibronectin polypeptide monobodies). Antibody fragments can be incorporated into single chain molecules comprising a pair of tandem Fv segments (VH-CH1-VH-CH1) which, together with complementary light chain polypeptides, form a pair of antigen-binding sites (Zapata et al., (1995) Protein Eng. 8:1057-1062; and U.S. Pat. No. 5,641,870).

The structures and locations of immunoglobulin variable domains, e.g., CDRs, may be defined using well known numbering schemes, e.g., the Kabat numbering scheme, the Chothia numbering scheme, or a combination of Kabat and Chothia (see, e.g. Sequences of Proteins of Immunological Interest, U.S. Department of Health and Human Services (1991), eds. Kabat et al.; Lazikani et al., (1997) J. Mol. Bio. 273:927-948); Kabat et al., (1991) Sequences of Proteins of Immunological Interest, 5th edit., NIH Publication no. 91-3242 U.S. Department of Health and Human Services; Chothia et al., (1987) J. Mol. Biol. 196:901-917; Chothia et al., (1989) Nature 342:877-883; and Al-Lazikani et al., (1997) J. Mol. Biol. 273:927-948; Annals of the New York Academy of Sciences, 764, 47-49 (1995); Nucleic Acids Research, 25, 206-211 (1997).

A “human antibody” or “human antibody fragment”, as used herein, is an antibody and antibody fragment having variable regions in which both the framework and CDR regions are from sequences of human origin. Human antibodies can also be isolated from synthetic libraries or from transgenic mice (e.g. Xenomouse) provided the respective system yield in antibodies having variable regions in which both the framework and CDR regions are derived from sequences of human origin. Furthermore, if the antibody contains a constant region, the constant region also is derived from such sequences. Human origin includes, e.g., human germline sequences, or mutated versions of human germline sequences or antibody containing consensus framework sequences derived from human framework sequences analysis, for example, as described in Knappik et al., (2000) J Mol Biol 296:57-86).

A “humanized antibody” or “humanized antibody fragment” is defined herein as an antibody molecule, which has constant antibody regions derived from sequences of human origin and the variable antibody regions or parts thereof or only the CDRs are derived from another species. For example, a humanized antibody can be CDR-grafted, wherein the CDRs of the variable domain are from a non-human origin, while one or more frameworks of the variable domain are of human origin and the constant domain (if any) is of human origin.

The term “chimeric antibody” or “chimeric antibody fragment” is defined herein as an antibody molecule, which has constant antibody regions derived from, or corresponding to, sequences found in one species and variable antibody regions derived from another species. Preferably, the constant antibody regions are derived from, or corresponding to, sequences found in humans, and the variable antibody regions (e.g. VH, VL, CDR or FR regions) are derived from sequences found in a non-human animal, e.g. a mouse, rat, rabbit or hamster.

The term “isolated antibody” refers to an antibody or antibody fragment that is substantially free of other antibodies or antibody fragments having different antigenic specificities. Moreover, an isolated antibody or antibody fragment may be substantially free of other cellular material and/or chemicals. Thus, in some aspects, antibodies provided are isolated antibodies, which have been separated from antibodies with a different specificity. An isolated antibody may be a monoclonal antibody. An isolated antibody may be a recombinant monoclonal antibody. An isolated antibody that specifically binds to an epitope, isoform or variant of a target may, however, have cross-reactivity to other related antigens, e.g., from other species (e.g., species homologs).

The term “recombinant antibody”, as used herein, includes all antibodies that are prepared, expressed, created or segregated by means not existing in nature. For example, antibodies isolated from a host cell transformed to express the antibody, antibodies selected and isolated from a recombinant, combinatorial human antibody library, and antibodies prepared, expressed, created or isolated by any other means that involve splicing of all or a portion of a human immunoglobulin gene, sequences to other DNA sequences or antibodies isolated from an animal (e.g., a mouse) that is transgenic or transchromosomal for human immunoglobulin genes or a hybridoma prepared therefrom. Preferably, such recombinant antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the VH and VL regions of the recombinant antibodies are sequences that, while derived from and related to human germline VH and VL sequences, may not naturally exist within the human antibody germline repertoire in vivo. A recombinant antibody may be a monoclonal antibody. In an embodiment, the antibodies and antibody fragment disclosed herein are isolated from the HuCAL library (Rothe et al, J. Mol. Biol. (2008) 376, 1 182-1200).

As used herein, an antibody “binds specifically to”, “specifically binds to”, is “specific to/for” or “specifically recognizes” an antigen, such as human CCR2, if such antibody is able to discriminate between such antigen and one or more reference antigen(s), since binding specificity is not an absolute, but a relative property. For example, a standard ELISA assay or standard flow cytometry assay can be carried out. The scoring may be carried out by standard color development (e.g. secondary antibody with horseradish peroxide and tetramethyl benzidine with hydrogen peroxide) or by binding of a secondary antibody labeled with PE or another dye or marker. The reaction in certain wells is scored by the optical density (OD), for example, at 450 nm or by mean fluorescence intensity (MFI) in flow cytometry. Typical background (=negative reaction) may be 0.1 OD; typical positive reaction may be 1 OD. Background and positive reaction MFI are highly dependent on instrument settings. The difference positive/negative can be more than 10-fold. Typically, determination of binding specificity is performed by using not a single reference antigen, but a set of about three to five unrelated antigens, such as milk powder, BSA, transferrin or the like. For flow cytometry various antigen-negative cells can be used. An antibody that specifically binds to an antigen may however have cross-reactivity to the respective orthologous antigen from other species (e.g., species homologs). In certain embodiments such cross-reactivity to an orthologous antigen is even preferred.

As used herein, an antibody has “cross-reactivity” or is “cross-reactive” if it binds to the orthologous antigen from other species. For example, an antibody is cross-reactive if it binds to human CCR2 and to marmoset CCR2.

As used herein, the term “affinity” refers to the strength of interaction between the polypeptide and its target at a single site. Within each site, the binding region of the polypeptide interacts through weak non-covalent forces with its target at numerous sites; the more interactions, the stronger the affinity.

The term “epitope” includes any proteinaceous region which is specifically recognized by an antibody or antibody fragment thereof or otherwise interacts with a molecule. Generally, epitopes are of chemically active surface groupings of molecules such as amino acids or carbohydrate or sugar side chains and generally may have specific three-dimensional structural characteristics, as well as specific charge characteristics. As will be appreciated by one of skill in the art, practically anything to which an antibody can specifically bind could be an epitope.

The term “domain” or “protein domain” refers to a region of a protein's polypeptide chain that forms a functional unit and/or independently forms a three-dimensional structure. For example the ECL2 of CCR2 is a protein domain.

“Compositions” of the present disclosure may be used for therapeutic or prophylactic applications. The present disclosure, therefore, includes a pharmaceutical composition containing an antibody or antibody fragment as disclosed herein and a pharmaceutically acceptable carrier or excipient therefore. In a related aspect, the present disclosure provides a method for treating inflammatory diseases, autoimmune diseases, hematologic malignancies and potentially other diseases. Such method contains the steps of administering to a subject in need thereof an effective amount of the pharmaceutical composition that contains an antibody or antibody fragment as described herein.

The present disclosure provides therapeutic methods comprising the administration of a therapeutically effective amount of an antibody or antibody fragment as disclosed herein to a subject in need of such treatment. A “therapeutically effective amount” or “effective amount”, as used herein, refers to the amount of a CCR2 antibody necessary to elicit the desired biological response. In accordance with the subject disclosure, the therapeutic effective amount is the amount of a CCR2 antibody necessary to treat and/or prevent a disease.

“Administered” or “administration” includes but is not limited to delivery of a drug by an injectable form, such as, for example, an intravenous, intramuscular, intradermal or subcutaneous route or mucosal route, for example, as a nasal spray or aerosol for inhalation or as an ingestible solution, capsule or tablet. Preferably, the administration is by an injectable form.

As used herein, “treatment”, “treat” or “treating” and the like refers to clinical intervention in an attempt to alter the natural course of a disease in the subject being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis. In some embodiments, antibodies or antibody fragments according to the preset disclosure are used to delay development of a disease or to slow the progression of a disease.

“Preventing” or “prevention” refers to a reduction in risk of acquiring or developing a disease (i.e. causing at least one of the clinical symptoms of the disease not to develop in a subject that may be exposed to a disease-causing agent, or predisposed to the disease in advance of disease onset). “Prevention” also refers to methods which aim to prevent the onset of a disease or its symptoms or which delay the onset of a disease or its symptoms.

“Subject” or “species” or as used in this context refers to any mammal, including rodents, such as mouse or rat, and primates, such as cynomolgus monkey (Macaca fascicularis), Marmoset monkey (Callithrix jacchus), rhesus monkey (Macaca mulatta) or humans (Homo sapiens). Preferably, the subject is a primate, most preferably a human.

The term “effector function” refers to those biological activities attributable to the Fc region of an antibody, which vary with the antibody isotype. Non-limiting examples of antibody effector functions include C1 q binding and complement dependent cytotoxicity (CDC); Fc receptor binding and antibody-dependent cell-mediated cytotoxicity (ADCC) and/or antibody-dependent cellular phagocytosis (ADCP); down regulation of cell surface receptors (e.g. B cell receptor); and direct cell activation or direct cell inhibition.

“Antibody-dependent cell-mediated cytotoxicity” or “ADCC” refers to a form of cytotoxicity in which antibodies bound onto Fc receptors (FcRs) present on certain cytotoxic cells (e.g. NK cells, neutrophils, and macrophages) enable these cytotoxic effector cells to bind specifically to an antigen-bearing target cell and subsequently kill the target cell with cytotoxins. The primary cells for mediating ADCC, NK cells, express FcγRIll only, whereas monocytes/macrophages express FcγRI, FcγRII, and FcγRIII.

“Complement-dependent cytotoxicity” or “CDC” refers to the lysis of a target cell in the presence of complement. Activation of the classical complement pathway is initiated by the binding of the first component of the complement system (C1q) to antibodies (of the appropriate subclass) of the present disclosure, which are bound to their cognate antigen.

“Antibody-dependent cellular phagocytosis” or “ADCP” refers to a mechanism of elimination of antibody-coated target cells by internalization by phagocytic cells, such as macrophages or dendritic cells.

Throughout this specification, unless the context requires otherwise, the words “comprise”, “have” and “include” and their respective variations such as “comprises”, “comprising”, “has”, “having”, “includes” and “including” will be understood to imply the inclusion of a stated element or integer or group of elements or integers but not the exclusion of any other element or integer or group of elements or integers.

The terms “engineered” or “modified” as used herein includes manipulation of nucleic acids or polypeptides by synthetic means (e.g. by recombinant techniques, in vitro peptide synthesis, by enzymatic or chemical coupling of peptides or some combination of these techniques). Preferably, the antibodies or antibody fragments according to the present disclosure are engineered or modified to improve one or more properties, such as antigen binding, stability, half-life, effector function, immunogenicity, safety and the like.

“Variant” as used herein refers to a polypeptide that differs from a reference polypeptide by one or more modifications for example amino acid substitutions, insertions or deletions. Variant polypeptides typically retain most of the properties of the reference polypeptide, e.g. binding to the target antigen, but introduce a novel, additional feature or property, e.g. the variant polypeptide has a higher affinity to the target antigen compared to the reference polypeptide or the variant polypeptide is a humanized version of the reference polypeptide.

The term “amino acid mutation” as used herein is meant to encompass amino acid substitutions, deletions, insertions, and modifications. Any combination of substitution, deletion, insertion, and modification can be made as long as the final construct possesses the desired characteristics, e.g., reduced binding to an Fc receptor. Amino acid sequence deletions and insertions include N- and/or C-terminal deletions and insertions of amino acid residues. Particular amino acid mutations are amino acid substitutions. Amino acid substitutions include replacement by non-naturally occurring amino acids or by naturally occurring amino acid derivatives of the twenty standard amino acids. Amino acid mutations can be generated using genetic or chemical methods well known in the art. Genetic methods may include site-directed mutagenesis, PCR, gene synthesis and the like. It is contemplated that methods of altering the side chain group of an amino acid residue by methods other than genetic engineering, such as chemical modification, may also be useful. Various designations may be used herein to indicate the same amino acid mutation. For example, a substitution from glycine at position 327 of the Fc region to alanine can be indicated as 237A, G337, G337A, or Gly329Ala.

The term “EC50” as used herein, refers to the concentration of an antibody or antibody fragment, which induces a response in an assay half way between the baseline and maximum. It therefore represents the antibody or ligand concentration at which 50% of the maximal effect is observed.

The terms “inhibition” or “inhibit” or “reduction” or “reduce” or “neutralization” or “neutralize” refer to a decrease or cessation of any phenotypic characteristic (such as binding or a biological activity or function) or to the decrease or cessation in the incidence, degree, or likelihood of that characteristic. “Inhibition”, “reduction” or “neutralization” needs not to be complete as long as it is detectable using an appropriate assay. In some embodiments, by “reduce” or “inhibit” or “neutralize” is meant the ability to cause a decrease of 20% or greater. In another embodiment, by “reduce” or “inhibit” or “neutralize” is meant the ability to cause a decrease of 50% or greater. In yet another embodiment, by “reduce” or “inhibit” or “neutralize” is meant the ability to cause an overall decrease of 75%, 85%, 90%, 95%, or greater.

The term “antagonistic” antibody as used herein refers to an antibody or antibody fragment that interacts with an antigen and partially or fully inhibits or neutralizes a biological activity or function or any other phenotypic characteristic of a target antigen.

A “wild-type” protein is a version or variant of the protein as it is found in nature. An amino acid sequence of a wildtype protein, e.g., a Fc region of an human IgG1 antibody, is the amino acid sequence of the protein as it occurs in nature. Due to allotypic differences, there can be more than one amino acid sequence for a wildtype protein. For example, there are several allotypes of naturally occurring human IGg1 heavy chain constant regions (see, e.g., Jeffries et al. (2009) mAbs 1:1).

The “Fc region” is used to define the C-terminal region of an immunoglobulin heavy chain. The Fc region of an immunoglobulin generally comprises two constant domains, a CH2 domain and a CH3 domain. Although the boundaries of the Fc region of an IgG heavy chain might vary slightly, the human IgG heavy chain Fc region is usually defined to extend from Cys226, or from Pro230, to the C-terminus of the heavy chain. However, the C-terminal lysine (Lys447) of the Fc region may or may not be present.

Unless otherwise specified herein, numbering of amino acid residues in the Fc region is according to the EU numbering system, also called the EU index, as described in Kabat et al., Sequences of Proteins of Immunological Interest, 5th Ed. Public Health Service, National Institutes of Health, Bethesda, MD, 1991.

### EMBODIMENTS OF THE INVENTION

### Polypeptides

In certain embodiments, the present disclosure relates to a humanized antibody or antibody fragment specific for human CCR2, wherein said antibody or antibody fragment comprises the HCDR1 region of SEQ ID NO: 2, the HCDR2 region of SEQ ID NO: 3, the HCDR3 region of SEQ ID NO: 4, the LCDR1 region of SEQ ID NO: 7, the LCDR2 region of SEQ ID NO: 8 and the LCDR3 region of SEQ ID NO: 9.

In certain embodiments, the present disclosure relates to a humanized antibody or antibody fragment specific for human CCR2, wherein said antibody or antibody fragment comprises the HCDR1 region of SEQ ID NO: 12, the HCDR2 region of SEQ ID NO: 13, the HCDR3 region of SEQ ID NO: 14, the LCDR1 region of SEQ ID NO: 17, the LCDR2 region of SEQ ID NO: 18 and the LCDR3 region of SEQ, ID NO: 19.

In certain embodiments, the present disclosure relates to a humanized antibody or antibody fragment specific for human CCR2, wherein said antibody or antibody fragment comprises the VH of SEQ ID NO: 21 and the VL of SEQ ID NO 23.

In certain embodiments, the present disclosure relates to a humanized antibody or antibody fragment specific for human CCR2, wherein said antibody or antibody fragment comprises the VH of SEQ ID NO: 25 and the VL of SEQ ID NO 29.

In certain embodiments, the present disclosure relates to a humanized antibody or antibody fragment specific for human CCR2, wherein said antibody or antibody fragment comprises the VH of SEQ ID NO: 27 and the VL of SEQ ID NO 29.

In certain embodiments, the present disclosure relates to a humanized antibody or antibody fragment specific for human CCR2, wherein said antibody or antibody fragment comprises the VH of SEQ ID NO: 21 and the VL of SEQ ID NO 31.

In certain embodiments, the present disclosure relates to a humanized antibody or antibody fragment specific for human CCR2, wherein said antibody or antibody fragment comprises the VH of SEQ ID NO: 33 and the VL of SEQ ID NO 23.

In certain embodiments, the present disclosure relates to a humanized antibody or antibody fragment specific for human CCR2, wherein said antibody or antibody fragment comprises the VH of SEQ ID NO: 33 and the VL of SEQ ID NO 31.

In certain embodiments, the present disclosure relates to a humanized antibody or antibody fragment specific for human CCR2, wherein said antibody or antibody fragment comprises the VH of SEQ ID NO: 33 and the VL of SEQ ID NO 35.

In certain embodiments, the present disclosure relates to a humanized antibody or antibody fragment specific for human CCR2, wherein said antibody or antibody fragment comprises the VH of SEQ ID NO: 37 and the VL of SEQ ID NO 29.

In certain embodiments, the present disclosure relates to a humanized antibody or antibody fragment specific for human CCR2, wherein said antibody or antibody fragment comprises the VH of SEQ ID NO: 37 and the VL of SEQ, ID NO 39.

In certain embodiments, the present disclosure relates to a humanized antibody or antibody fragment specific for human CCR2, wherein said antibody or antibody fragment comprises the VH of SEQ ID NO: 21 and the VL of SEQ ID NO 41.

In certain embodiments, the present disclosure relates to a humanized antibody or antibody fragment specific for human CCR2, wherein said antibody or antibody fragment comprises the VH of SEQ ID NO: 21 and the VL of SEQ ID NO 43.

In certain embodiments, the present disclosure relates to a humanized antibody or antibody fragment specific for human CCR2, wherein said antibody or antibody fragment comprises the VH of SEQ ID NO: 21 and the VL of SEQ ID NO 45.

In certain embodiments, the present disclosure relates to a humanized antibody or antibody fragment specific for human CCR2, wherein said antibody or antibody fragment comprises the VH of SEQ ID NO: 21 and the VL of SEQ ID NO 47.

In certain embodiments, the humanized antibodies specific for CCR2 are of the IgG1 class. In certain embodiments, the constant heavy domain is of the G1m3 allotype. In certain embodiments, the humanized antibodies comprise the amino acid sequence of SEQ ID No. 49. In certain embodiments, the constant light domain is of the Km3 allotype. In certain embodiments, the humanized antibodies comprise the amino acid sequence of SEQ ID No. 51.

In certain embodiments, the present disclosure relates to a humanized antibody specific for human CCR2, wherein said antibody comprises the VH of SEQ ID NO: 21 and the VL of SEQ ID NO 43, and wherein said humanized antibody is of the IgG1 isotype.

In certain embodiments, the present disclosure relates to a humanized antibody specific for human CCR2, wherein said antibody comprises the VH of SEQ ID NO: 21 and the VL of SEQ ID NO 43, and wherein said humanized antibody is of the G1m3 allotype.

In certain embodiments, the present disclosure relates to a humanized antibody specific for human CCR2, wherein said antibody comprises the VH of SEQ ID NO: 21 and the VL of SEQ ID NO 43, and wherein said humanized antibody is of the Km3 allotype.

In certain embodiments, the present disclosure relates to a humanized antibody specific for human CCR2, wherein said antibody comprises the VH of SEQ ID NO: 21 and the VL of SEQ ID NO 43, and wherein said humanized antibody is of the G1m3 allotype and the Km3 allotype.

In certain embodiments, the present disclosure relates to a humanized antibody or antibody fragment, wherein said antibody or antibody fragment binds to the first extracellular loop of human CCR2 (SEQ ID No. 53) and the second extracellular loop of human CCR2 (SEQ ID No. 54).

In certain embodiments, the present disclosure relates to a humanized antibody or antibody fragment, wherein said antibody or antibody fragment binds to the first extracellular loop of marmoset CCR2 (SEQ ID No. 53) and the second extracellular loop of marmoset CCR2 (SEQ ID No. 55).

In certain embodiments, the present disclosure relates to a humanized antibody or antibody fragment, wherein said antibody or antibody fragment is cross-reactive with marmoset CCR2.

In certain embodiments, the present disclosure relates to a humanized antibody or antibody fragment, wherein said antibody or antibody fragment does not bind to rhesus CCR2.

In certain embodiments, the present disclosure relates to a humanized antibody or antibody fragment, wherein said antibody or antibody fragment specifically binds to a polypeptide comprising the amino acid sequence of SEQ ID No. 53 and a polypeptide comprising the amino acid sequence of SEQ ID No. 54.

In certain embodiments, the present disclosure relates to a humanized antibody or antibody fragment, wherein said antibody or antibody fragment specifically binds to a polypeptide comprising the amino acid sequence of SEQ ID No. 53 and a polypeptide comprising the amino acid sequence of SEQ ID No. 54, but not to a polypeptide comprising the amino acid sequence of SEQ ID No. 55.

In certain embodiments, the present disclosure relates to a humanized antibody or antibody fragment specific for human CCR2, wherein said antibody or antibody fragment comprises the VH of SEQ ID NO: 21 and the VL of SEQ ID NO 43, wherein said antibody or antibody fragment binds to the first extracellular loop of human CCR2 (SEQ ID No. 53) and the second extracellular loop of human CCR2 (SEQ ID No. 54).

In certain embodiments, the present disclosure relates to a humanized antibody or antibody fragment specific for human CCR2, wherein said antibody or antibody fragment comprises the VH of SEQ ID NO: 21 and the VL of SEQ ID NO 43, and wherein said antibody or antibody fragment binds to the first extracellular loop of marmoset CCR2 (SEQ ID No. 53) and the second extracellular loop of marmoset CCR2 (SEQ ID No. 55).

In certain embodiments, the present disclosure relates to a humanized antibody or antibody fragment specific for human CCR2, wherein said antibody or antibody fragment comprises the VH of SEQ ID NO: 21 and the VL of SEQ ID NO 43, and wherein said antibody or antibody fragment is cross-reactive with marmoset CCR2.

In certain embodiments, the present disclosure relates to a humanized antibody or antibody fragment specific for human CCR2, wherein said antibody or antibody fragment comprises the VH of SEQ ID NO: 21 and the VL of SEQ ID NO 43, and wherein said antibody or antibody fragment does not bind to rhesus CCR2.

In certain embodiments, the present disclosure relates to a humanized antibody or antibody fragment specific for human CCR2, wherein said antibody or antibody fragment comprises the VH of SEQ ID NO: 21 and the VL of SEQ ID NO 43, and wherein said antibody or antibody fragment specifically binds to a polypeptide comprising the amino acid sequence of SEQ ID No. 53 and a polypeptide comprising the amino acid sequence of SEQ ID No. 54.

In certain embodiments, the present disclosure relates to a humanized antibody or antibody fragment specific for human CCR2, wherein said antibody or antibody fragment comprises the VH of SEQ ID NO: 21 and the VL of SEQ ID NO 43, and wherein said antibody or antibody fragment specifically binds to a polypeptide comprising the amino acid sequence of SEQ ID No. 53 and a polypeptide comprising the amino acid sequence of SEQ ID No. 54, but not to a polypeptide comprising the amino acid sequence of SEQ ID No. 55.

### Nucleic Acids, Vectors and Host Cells

In certain embodiments, the present disclosure relates to nucleic acids encoding the humanized antibodies or antibody fragments specific for human CCR2 disclosed herein. In certain embodiments, the present disclosure relates to a nucleic acid encoding the humanized antibodies or antibody fragments specific for human CCR2 disclosed herein. In certain embodiments, the present disclosure relates to a plurality of nucleic acid sequences encoding the humanized antibodies or antibody fragments specific for human CCR2 disclosed herein.

In certain embodiments, the present disclosure relates to a nucleic acid encoding a humanized antibody or antibody fragment specific for human CCR2 disclosed, wherein said humanized antibody or antibody fragment comprises a variable heavy chain comprising the amino acid sequence of SEQ ID No. 21 and a variable light chain comprising the amino acid sequence of SEQ ID No. 43.

In certain embodiments, the present disclosure relates to a nucleic acid encoding a humanized antibody or antibody fragment specific for human CCR2 disclosed, wherein said humanized antibody or antibody fragment comprises a variable heavy chain consisting of the amino acid sequence of SEQ ID No. 21 and a variable light chain consisting of the amino acid sequence of SEQ ID No. 43.

In certain embodiments, the present disclosure relates to a plurality of nucleic acids encoding a humanized antibody or antibody fragment specific for human CCR2 disclosed, wherein said humanized antibody or antibody fragment comprises a variable heavy chain comprising the amino acid sequence of SEQ ID No. 21 and a variable light chain comprising the amino acid sequence of SEQ ID No. 43.

In certain embodiments, the present disclosure relates to a plurality of nucleic acids encoding a humanized antibody or antibody fragment specific for human CCR2 disclosed, wherein said humanized antibody or antibody fragment comprises a variable heavy chain consisting of the amino acid sequence of SEQ ID No. 21 and a variable light chain consisting of the amino acid sequence of SEQ ID No. 43.

In certain embodiments, the present disclosure relates to a nucleic acid encoding a humanized antibody or antibody fragment specific for human CCR2 disclosed, wherein nucleic acid comprises the nucleic acid sequence of SEQ ID No. 22 and the nucleic acid sequence of SEQ ID No. 44.

In certain embodiments, the present disclosure relates to a plurality of nucleic acids encoding a humanized antibody or antibody fragment specific for human CCR2 disclosed, wherein nucleic acids comprise the nucleic acid sequences of SEQ ID No. 22 and SEQ ID No. 44.

In certain embodiments, the present disclosure relates to a vectors comprising a nucleic acid composition comprising a nucleic acid sequence or a plurality of nucleic acid sequences encoding a humanized antibody or antibody fragment specific for human CCR2 according to the present disclosure.

In certain embodiments, the present disclosure relates to a host cell comprising a vector composition comprising a vector or a plurality of vectors comprising a nucleic acid composition comprising a nucleic acid sequence or a plurality of nucleic acid sequences encoding a humanized antibody or antibody fragment specific for human CCR2 according to the present disclosure. In an embodiment, the present disclosure relates to a host cell comprising a vector composition.

In an embodiment, the host cell according to the present disclosure is able to express the humanized antibodies or antibody fragments specific for human CCR2 encoded by the vector composition or the nucleic acid composition.

In a further embodiment, the host cell is an isolated host cell. In a further embodiment, said host cell is a mammalian cell. In an embodiment, said mammalian cell is a human cell. In another embodiment, said mammalian cell is a CHO cell. In an embodiment, said cell is a HEK cell. In another embodiment, said cell is a PERC.6 cell. In an embodiment, said cell is a HKB11 cell.

The skilled person will realize that the nucleic acid sequence or the plurality of nucleic acid sequences encoding the heavy and/or light chain of an antibody or antibody fragment of the present disclosure can be cloned into different vectors or into the same vector.

The vectors can be introduced into the appropriate host cells such as prokaryotic (e.g., bacterial) or eukaryotic (e.g., yeast or mammalian) cells by methods well known in the art (see e.g., “Current Protocol in Molecular Biology”, Ausubel et al. (eds.), Greene Publishing Assoc and John Wiley Interscience, New York, 1989 and 1992). Numerous cloning vectors are known to those of skill in the art, and the selection of an appropriate cloning vector is a matter of choice. The gene can be placed under the control of a promoter, ribosome binding site (for bacterial expression) and, optionally, an operator (collectively referred to herein as “control” elements), so that the nucleic acid sequence encoding the desired protein is transcribed into RNA in the host cell transformed by a vector containing this expression construction. The coding sequence may or may not contain a signal peptide or leader sequence. Upon expression in host cells, the antibodies or antibody fragments of the present disclosure are obtained. These steps can be achieved in different ways, as will be known by the person skilled in the art. In general, such steps typically include transforming or transfecting a suitable host cell with a nucleic acid composition or vector composition or an infectious particle, which encodes the antibody, or antibody fragments. Further, such steps typically include culturing said host cells under conditions suitable for the proliferation (multiplication, growth) of said host cells and a culturing step under conditions suitable for the production (expression, synthesis) of the encoded antibody or antibody fragment. The culturing of host cells under conditions suitable for proliferation or expression is typically accomplished in the presence of media comprising components suitable for cell growth or induction of expression. In particular, embodiments, the methods for the production of the antibodies or antibody fragments of the present disclosure further comprise the step of isolating and purifying the produced antibody or antibody fragment from the host cells or medium. If the expression system secretes the protein into growth media, the protein can be purified directly from the media. If the protein is not secreted, it is isolated from cell lysates or recovered from the cell membrane fraction. The selection of the appropriate growth conditions and recovery methods are within the skill of the art. The antibody or antibody fragment of the present disclosure can then be purified by a number of techniques as known to the person skilled in the art.

In certain embodiments, the present disclosure refers to a method of producing a humanized antibody or antibody fragment specific for human CCR2 according to the present disclosure. In certain embodiments, a method of producing a humainzed antibody or antibody fragment according to the present disclosure is provided, wherein the method comprises culturing a host cell comprising a vector composition comprising a vector or a plurality of vectors comprising a nucleic acid composition comprising a nucleic acid sequence or a plurality of nucleic acid sequences encoding a humanized antibody or antibody fragment according to the present disclosure, under conditions suitable for expression of said humanized antibody or antibody fragment, and isolating said humanized antibody or antibody fragment from the host cell or host cell culture medium.

The humanized antibodies or antibody fragments disclosed herein may be purified techniques know in the art, such as high performance liquid chromatography (HPLC), ion exchange chromatography, gel electrophoresis, affinity chromatography, size exclusion chromatography, and the like. The conditions used to purify a particular antibody or antibody fragment will depend, in part, on factors such as net charge, hydrophobicity, hydrophilicity etc., and will be apparent to those having skill in the art. For affinity chromatography purification an antibody, ligand, receptor or antigen can be used to which the antibody or antibody fragment binds. For example, for affinity chromatography purification of antibody or antibody fragment according to the present disclosure, a matrix with protein A or protein G may be used. The purity of an antibody or antibody fragment can be determined by any of a variety of well-known analytical methods including gel electrophoresis, high-pressure liquid chromatography, and the like.

### Therapeutic Use

In an embodiment, the disease is associated with the undesired presence of CCR2, in human CCR2. In another embodiment, the disease is associated with the undesired presence of CCR2 positive cells, in particular human CCR2 positive cells. Human CCR2 positive cells include CD16-negative monocytes, basophils and plasmacytoid dendritic cells. Also about 20% of CD4+ and CD8+ T cells express CCR2.

In an embodiment, the disease to be treated is an autoimmune or inflammatory disease. Non-limiting examples an autoimmune or inflammatory disease include rheumatoid arthritis (RA), psoriasis, psoriatic arthritis, systemic lupus erythematosus (SLE), lupus nephritis, type I diabetes, Grave's disease, Inflammatory bowel disease (IBD), Crohn's disease (CD), ulcerative colitis (UC), irritable bowel syndrome, multiple sclerosis (MS), Guillain-Barre's Syndrome, autoinflammatory diseases like Familial Mediterranean Fever (FMF), Cryopyrin-associated periodic syndromes (CAPS), Deficiency of IL-1-Receptor Antagonist (DIRA), Hyper IgD Syndrome (HIDS), autoimmune myocarditis, Kawasaki disease, coronary artery disease, chronic obstructive pulmonary disease (COPD), interstitial lung disease, autoimmune thyroiditis, scleroderma, systemic sclerosis, osteoarthritis, atoptic dermatitis, vitiligo, graft vs. host disease, Sjogren's syndrome, autoimmune nephritis, Goodpasture's syndrome, chronic inflammatory demyelinating polyneuropathy, ANCA-associated vasculitis, uveitis, scleroderma, bullous pemphigoid, Alzheimer's Disease, amyotrophic lateral sclerosis, Huntington's Chorea, cystic fibrosis, gout, age-related macular degeneration, allergy, asthma, antiphospholipid syndrome (APS), atherosclerosis, C3 glomerulopathy and IgA nephropathy, ischemia/reperfusion injury, peritonitis, sepsis and other autoimmune diseases that are a result of either acute or chronic inflammation.

In an embodiment, the disease to be treated is a proliferative disease. In a particular embodiment, the disease is cancer. Non-limiting examples of cancers include hematologic malignancies like chronic myelomonocytic leukemia (CMML), acute myeloid leukemia (AML), myelodysplastic syndrome, mastocytosis and non-hematologic malignancies like bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, blood cancer, sarcoma, skin cancer, squamous cell carcinoma, bone cancer, melanoma, renal cell carcinoma, and kidney cancer.

In an embodiment, the present disclosure provides a method for the treatment of a disease.

In an embodiment, the present disclosure provides a method for the treatment of a disease comprising administering to a patient a humanized antibody or antibody fragment of the present disclosure.

In an embodiment, the present disclosure provides a method for the treatment of a disease comprising administering to a subject in need there of a humanized antibody or antibody fragment of the present disclosure.

In an embodiment, the present disclosure provides a method for the prevention of a disease.

In an embodiment, the present disclosure provides a method for the prevention of a disease comprising administering to a subject a humanized antibody or antibody fragment of the present disclosure.

In an embodiment, the present disclosure provides a humanized antibody or antibody fragment according to the present disclosure for the treatment of a disease. In an embodiment, the present disclosure provides a humanized antibody or antibody fragment according to the present disclosure for use in the treatment of a disease. In an embodiment, the present disclosure provides a humanized antibody or antibody fragment according to the present disclosure for use in the treatment of a disease in a subject in need thereof.

In an embodiment, the present disclosure provides the use of a humanized antibody or antibody fragment according to the present disclosure for the manufacture of a medicament. In an embodiment, the present disclosure provides a huanized antibody or antibody fragment according to the present disclosure for use as a medicament. In an embodiment, the present disclosure provides a humanized antibody or antibody fragment according to the present disclosure for use in medicine. In an embodiment, the present disclosure provides a humanized antibody or antibody fragment according to the present disclosure for use as a medicament for the treatment of a subject in need thereof.

In an embodiment, the present disclosure provides a humanized isolated antibody or antibody fragment specific for human CCR2 according to the present disclosure for use in a method of treating a subject having a disease comprising administering to the subject a therapeutically effective amount of a humanized antibody or antibody fragment according to the present disclosure.

In an embodiment, the method further comprises administering to the subject a therapeutically effective amount of at least one additional therapeutic agent. The subject in need of treatment is typically a mammal, more specifically a human. For use in therapeutic methods, an isolated antibody or antibody fragment according to the present disclosure would be formulated, dosed, and administered in a way consistent with good medical practice.

The humanized antibodies and antibody fragments according to the present disclosure may be used in therapeutic methods. The antibody or antibody fragment according to the present disclosure may be used for the treatment of inflammatory diseases, autoimmune diseases, hematologic malignancies and potentially other diseases.

In certain embodiments, the present disclosure relates to relates to a humanized antibody or antibody fragment specific for human CCR2 for use in medicine, wherein said antibody or antibody fragment comprises the HCDR1 region of SEQ ID NO: 2, the HCDR2 region of SEQ ID NO: 3, the HCDR3 region of SEQ ID NO: 4, the LCDR1 region of SEQ ID NO: 7, the LCDR2 region of SEQ ID NO: 8 and the LCDR3 region of SEQ ID NO: 9.

In certain embodiments, said use in medicine is the treatment of an inflammatory disease, an autoimmune diseases or a hematologic malignancy. Therefore in certain embodiments, the present disclosure relates to relates to a humanized antibody or antibody fragment specific for human CCR2 for use in the treatment of an inflammatory disease, an autoimmune diseases or a hematologic malignancy, wherein said antibody or antibody fragment comprises the HCDR1 region of SEQ ID NO: 2, the HCDR2 region of SEQ ID NO: 3, the HCDR3 region of SEQ ID NO: 4, the LCDR1 region of SEQ ID NO: 7, the LCDR2 region of SEQ ID NO: 8 and the LCDR3 region of SEQ ID NO: 9.

In certain embodiments, the present disclosure relates to relates to a humanized antibody or antibody fragment specific for human CCR2 for use in medicine, wherein said antibody or antibody fragment comprises the VH of SEQ ID NO: 21 and the VL of SEQ ID NO 43.

In certain embodiments, the present disclosure relates to relates to a humanized antibody or antibody fragment specific for human CCR2 for use in the treatment of an inflammatory disease, an autoimmune diseases or a hematologic malignancy, wherein said antibody or antibody fragment comprises the VH of SEQ ID NO: 21 and the VL of SEQ ID NO 43.

In certain embodiments, the present disclosure relates to relates to a humanized antibody or antibody fragment specific for human CCR2 for use in the treatment of an inflammatory disease, wherein said antibody or antibody fragment comprises the VH of SEQ ID NO: 21 and the VL of SEQ ID NO 43.

In certain embodiments, the present disclosure relates to relates to a humanized antibody or antibody fragment specific for human CCR2 for use in the treatment of an autoimmune diseases, wherein said antibody or antibody fragment comprises the VH of SEQ ID NO: 21 and the VL of SEQ, ID NO 43.

In certain embodiments, the present disclosure relates to relates to a humanized antibody or antibody fragment specific for human CCR2 for use in the treatment of a hematologic malignancy, wherein said antibody or antibody fragment comprises the VH of SEQ ID NO: 21 and the VL of SEQ ID NO 43.

In certain embodiments, the present disclosure related to a method for the treatment of a disease or disorder comprising the administration of a humanized antibody or antibody fragment comprising the VH of SEQ ID NO: 21 and the VL of SEQ ID NO 43 to a patient in need thereof.

In certain embodiments, the present disclosure related to a method for the treatment of an inflammatory disease, an autoimmune diseases or a hematologic malignancy comprising the administration of a humanized antibody or antibody fragment comprising the VH of SEQ ID NO: 21 and the VL of SEQ ID NO 43 to a patient in need thereof.

### Pharmaceutical Compositions

In certain embodiments, the present disclosure provides a pharmaceutical composition comprising a humanized antibody or antibody fragment according to the present disclosure and a pharmaceutically acceptable carrier or excipient.

The pharmaceutical compositions may further comprise at least one other pharmaceutically active compound. The pharmaceutical composition according to the present disclosure can be used in the diagnosis, prevention and/or treatment of diseases associated with the undesired presence of CCR2, in particular human CCR2. The pharmaceutical composition according to the present disclosure can be used in the diagnosis, prevention and/or treatment of diseases associated with the undesired presence of CCR2 positive cells, in particular CCR2 positive human cells. In particular, the present disclosure provides a pharmaceutical compositions comprising a humanized antibody or antibody fragment according to the present disclosure that is suitable for prophylactic, therapeutic and/or diagnostic use in a mammal, more particular in a human.

In general, an antibody or antibody fragment according to the present disclosure may be formulated as a pharmaceutical composition comprising at least one antibody or antibody fragment according to the present disclosure and at least one pharmaceutically acceptable carrier or excipient, and optionally one or more further pharmaceutically active compounds. Such a formulation may be suitable for oral, parenteral, topical administration or for administration by inhalation. Accordingly, a pharmaceutical composition comprising at least one antibody or antibody fragment according to the present disclosure may be administered parenterally, such as intravenously, or intramuscularly, or subcutaneously. Alternatively, an antibody of the invention may be administered via a non-parenteral route, such as per-orally or topically. In a preferred embodiment, a pharmaceutical composition comprising an antibody or antibody fragment according to the present disclosure is administered intravenously or subcutaneously.

In particular, an antibody or antibody fragment according to the present disclosure may be used in combination with one or more pharmaceutically active compounds that are or can be used for the prevention and/or treatment of the diseases in which a target antigen of interest is involved, as a result of which a synergistic effect may or may not be obtained. Examples of such compounds, as well as routes, methods and pharmaceutical formulations or compositions for administering them will be clear to the clinician.

In certain embodiments, the present disclosure provides a pharmaceutical composition comprising a humanized antibody or antibody fragment according to the present disclosure for use in the prevention and/or treatment of a disease associated with the undesired presence of CCR2, in particular human CCR2. In an embodiment, the present disclosure provides a pharmaceutical composition comprising a humanized antibody or antibody fragment according to the present disclosure for use in the prevention and/or treatment of a disease associated with the undesired presence of CCR2 positive cells, in particular CCR2 positive human cells. In an embodiment, the present disclosure provides a pharmaceutical composition comprising a humanized antibody or antibody fragment according to the present disclosure for the use as a medicament. In an embodiment, the present disclosure provides a pharmaceutical composition comprising a humanized antibody or antibody fragment according to the present disclosure for use in the prevention and/or treatment of an autoimmune disease and/or inflammatory disease and/or cancer.

In certain embodiments, the present disclosure provides a method for the treatment of an autoimmune disease and/or inflammatory disease and/or cancer in a subject in need thereof using a pharmaceutical composition comprising a humanized antibody or antibody fragment according to the present disclosure.

Further provided is a method of producing a humanized antibody or antibody fragment according to the present disclosure in a form suitable for administration in vivo, the method comprising (a) obtaining a humanized antibody or antibody fragment by a method according to the present disclosure, and (b) formulating said humanized antibody or antibody fragment with at least one pharmaceutically acceptable carrier or excipient, whereby a preparation of said humanized antibody or antibody fragment is formulated for administration in vivo, Pharmaceutical compositions according to the present disclosure comprise a therapeutically effective amount of one or more humanized antibodies or antibody fragments according to the present disclosure dissolved in a pharmaceutically acceptable carrier or excipient.

### Diagnostic Use

In certain embodiments, the present disclosure provides the use of a humanized antibody or antibody fragment specific for human CCR2 according to the present disclosure for the diagnosis of a disease. In an embodiment, the present disclosure provides the use of a humanized antibody or antibody fragment according to the present disclosure for the detection of CCR2, in particular human CCR2 and/or marmoset CCR2. In an embodiment, the present disclosure provides a method for detecting CCR2 in a subject or a sample, comprising the step of contacting said subject or sample with a humanized antibody or antibody fragment specific for human CCR2 of the present disclosure. In an embodiment, the present disclosure provides a method for diagnosing a disease in a subject, comprising the step of contacting said subject or sample with a humanized antibody or antibody fragment according to the present disclosure. The antibodies may also be used to determine CCR2 expression levels in cells from patients. The CCR2 expressions levels may serve as therapeutic biomarkers, for example for patient stratification.

### EXAMPLES

### Example 1: Generation of Anti-CCR2 Antibodies Using Hybridoma Technology

Anti-CCR2 antibodies were generated using classical hybridoma technology. In brief, BALB/c mice were immunized with CHO cells stably transfected with human CCR2. Splenocytes were fused to X63Ag8 myeloma cells and hybridomas were screened for specific binding to CCR2-transfected CHO cells and absence of binding to CCR5-transfected CHO cells. Hybridomas were cloned and recloned by limited dilution technology.

Out of these antibodies only DOC2 and DOC3 showed cross-reactivity with marmoset CCR2, as tested with primary marmoset monocytes. Furthermore, antibodies DOC2 and DOC3 showed a downmodulation of CCR2 on marmoset monocytes by MCP-1.

Amino acid and nucleic acid sequences of antibodies DOC2 and DOC3 are shown in the following Table (CDRs according to Kabat).

TABLE 1

| SEQ | | |
|---|---|---|
| ID | | |
| No. | Description | Sequence |
| 3 | VH (DOC2) | EVQLVESGGGLVKPGGSLKLSCVASGFTLSNYAMSWVRQSP |
| | | EKRLEWVAEVSSSGIYIYYSDTVTGRFSISRDNAKNTLYLE |
| | | MSSLRSEDTAIYYCARDRYAYAMDYWGHGTSVIVSS |
| 2 | HCDR1 (DOC2) | NYAMS |
| 3 | HCDR2 (DOC2) | EVSSSGIYIYYSDTVTG |
| 4 | HCDR3 (DOC2) | DRYAYAMDY |
| | VH (DOC2) | GAGGTGCAGCTGGTGGAATCCGGCGGAGGCCTGGTGAAACC |
| | | TGGCGGCTCCCTGAAGCTGTCCTGCGTGGCCTCCGGCTTCA |
| | | CCCTGTCCAACTACGCCATGTCCTGGGTGCGACAGTCCCCC |
| | | GAGAAGCGGCTGGAATGGGTGGCCGAGGTCTCCTCCTCCGG |
| | | CATCTACATCTACTACTCCGACACCGTGACAGGCCGGTTCT |
| | | CCATCTCCCGGGACAACGCCAAGAACACCCTGTACCTGGAA |
| | | ATGTCTAGCCTGCGGAGCGAGGACACCGCTATCTACTACTG |
| | | CGCCAGAGACAGATACGCCTACGCCATGGACTACTGGGGCC |
| | | ACGGCACCTCCGTGATCGTGTCCTCTGC |
| 6 | VL (DOC2) | DIVMTQSPSSLAISVGQRVTLSCKSSQSLLNSYNQKNSLAW |
| | | YQQKPGQSPKLLVYFASTRESGVPDRFIGSGSGTYFTLTIT |
| | | SVQAGDLADYFCQQHYSNPRIFGGGTRLEIK |
| 7 | LCDR1 (DOC2) | KSSQSLLNSYNQKNSLA |
| 8 | LCDR2 (DOC2) | FASTRES |
| 9 | LCDR3 (DOC2) | QQHYSNPRT |
| 10 | VL (DOC2) | GACATCGTGATGACCCAGTCCCCCTCCAGCCTGGCCATCTC |
| | | CGTGGGCCAGAGAGTGACCCTGTCCTGCAAGTCCTCCCAGT |
| | | CCCTGCTGAACTCCTACAACCAGAAGAACTCCCTGGCCTGG |
| | | TATCAGCAGAAGCCCGGCCAGTCCCCCAAGCTGCTGGTGTA |
| | | CTTCGCCTCCACCCGCGAGTCCGGCGTGCCCGACAGATTCA |
| | | TCGGCTCCGGCTCCGGCACCTACTTCACCCTGACCATCACC |
| | | TCCGTGCAGGCCGGCGACCTGGCCGACTACTTCTGCCAGCA |
| | | GCACTACTCCAACCCCCGGACCTTCGGCGGAGGCACCCGGC |
| | | TGGAAATCAAG |
| 11 | VH (DOC3) | QVQLKESGPGLVAPSQSLSITCTVSGESLTRYDLSWIRQPP |
| | | GKGLQWLGVIWTGGGTNYNSAFMSRLSIRKDNSKSQVFLKM |
| | | NSLQTDDTAIYYCVRENYYNRRGYFDVWGAGTTVTVSS |
| 12 | HCDR1 (DOC3) | RYDLS |
| 13 | HCDR2 (DOC3) | VIWTGGGTNYNSAFMS |
| 14 | HCDR3 (DOC3) | ENYYNRRGYFDV |
| 15 | VH (DOC3) | CAGGTGCAGCTGAAAGAGTCCGGCCCTGGCCTGGTGGCCCC |
| | | TTCCCAGTCCCTGTCCATCACCTGTACCGTGTCCGGCTTCA |
| | | GCCTGACCAGATACGACCTGTCCTGGATCAGACAGCCCCCT |
| | | GGCAAGGGCCTGCAGTGGCTGGGAGTGATCTGGACCGGCGG |
| | | AGGCACCAACTACAACTCCGCCTTCATGTCCCGGCTGTCCA |
| | | TCAGAAAGGACAACTCCAAGTCCCAGGTGTTCCTGAAGATG |
| | | AACTCCCTGCAGACCGACGACACCGCCATCTACTACTGCGT |
| | | GCGCGAGAACTACTACAACAGACGGGGCTACTTCGACGTGT |
| | | GGGGCGCTGGCACCACCGTGACAGTGTCCTCT |
| 16 | VL (DOC3) | QIVLIQSPAIMSTSPGEKVTITCSASSSVSYMHWFQQKPGT |
| | | SPKLWIYLTSNLASGVPARFSGSGSGTSYSLTINRMEAEDA |
| | | ATYYCQQRSGSPPTFGTGTKLELR |
| 17 | LCDRI (DOC3) | SASSSVSYMH |
| 18 | LCDR2 (DOC3) | LTSNLAS |
| 19 | LCDR3 (DOC3) | QQRSGSPPT |
| 20 | VL (DOC3) | CAGATCGTGCTGACCCAGTCCCCCGCCATCATGTCCACCAG |
| | | CCCTGGCGAGAAAGTGACAATCACCTGTTCCGCCAGCTCCT |
| | | CCGTGTCCTACATGCACTGGTTCCAGCAGAAGCCCGGCACC |
| | | TCCCCCAAGCTGTGGATCTACCTGACCTCCAACCTGGCCTC |
| | | CGGCGTGCCCGCCAGATTCTCTGGCTCCGGCTCCGGCACCA |
| | | GCTACTCCCTGACCATCAACCGGATGGAAGCCGAGGACGCC |
| | | GCCACCTACTACTGCCAGCAGAGATCCGGCTCCCCCCCTAC |
| | | CTTCGGCACCGGCACCAAACTGGAACTGCGG |

### Example 2: Generation of Chimeric Versions of Antibodies DOC2 and DOC3

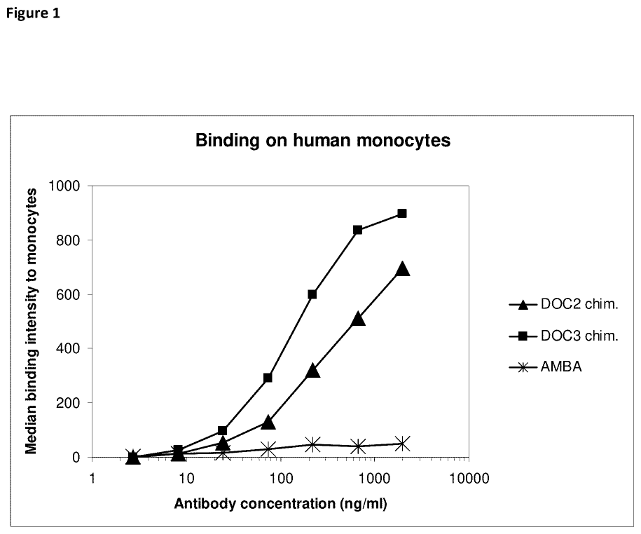

Chimeric antibodies containing human IgG1, kappa constant domains were generated from antibodies DOC2 and DOC3. Both chimeric antibodies, DOC-2 chim, and DOC3 chim., showed excellent binding to human and marmoset CCR2 using primary human and marmoset PBMC. Results are shown in FIG. 1. It was found that antibody DOC3 chim. showed a slightly better binding to human and marmoset PBMC than DOC2 chim. The antibody AMBA (provided by LifeArc/MRCT) served as negative control.

Amino acid sequences and nucleic acid sequences of the constant regions of the chimeric variants of DOC2 and DOC3, as well as of the humanized variants of the following examples, are as follows.

TABLE 2

| SEQ ID No. | Description | Sequence |
|---|---|---|
| 49 | CH (IgG1, G1m3 | ASTKGPSVFPLAPSSKSTSGGTAALGCLVKDYFPEPVTVSW |
| | allotype) | NSGALTSGVHTFPAVLQSSGLYSLSSVVTVPSSSLGTQTYI |
| | | CNVNHKPSNTKVDKRVEPKSCDKTHTCPPCPAPELLGGPSV |
| | | FLFPPKPKDTLMISRTPEVTCVVVDVSHEDPEVKENWYVDG |
| | | VEVHNAKIKPREEQYNSTYRVVSVLIVLHQDWLNGKEYKCK |
| | | VSNKALPAPIEKTISKAKGQPREPQVYTLPPSREEMTKNQV |
| | | SLTCLVKGFYPSDIAVEWESNGQPENNYKTTPPVLDSDGSF |
| | | FLYSKLTVDKSRWQQGNVFSCSVMHEALHNHYTQKSLSLSP |
| | | GK |
| 50 | CH (IgG1, G1m3 | GCCTCCACCAAGGGCCCATCGGTCTTCCCCCTGGCACCCTC |
| | allotype) | CTCCAAGAGCACCTCTGGGGGCACAGCGGCCCTGGGCTGCC |
| | | TGGTCAAGGACTACTTCCCCGAACCGGTGACGGTGTCGTGG |
| | | AACTCAGGCGCCCTGACCAGCGGCGTGCACACCTTCCCGGC |
| | | TGTCCTACAGTCCTCAGGACTCTACTCCCTCAGCAGCGTGG |
| | | TGACCGTGCCCTCCAGCAGCTTGGGCACCCAGACCTACATC |
| | | TGCAACGTGAATCACAAGCCCAGCAACACCAAGGTGGACAA |
| | | GAGAGTTGAGCCCAAATCTTGTGACAAAACTCACACATGCC |
| | | CACCGTGCCCAGCACCTGAACTCCTGGGGGGACCGTCAGTC |
| | | TTCCTCTTCCCCCCAAAACCCAAGGACACCCTCATGATCTC |
| | | CCGGACCCCTGAGGTCACATGCGTGGTGGTGGACGTGAGCC |
| | | ACGAAGACCCTGAGGTCAAGTTCAACTGGTACGTGGACGGC |
| | | GTGGAGGTGCATAATGCCAAGACAAAGCCGCGGGAGGAGCA |
| | | GTACAACAGCACGTACCGGGTGGTCAGCGTCCTCACCGTCC |
| | | TGCACCAGGACTGGCTGAATGGCAAGGAGTACAAGTGCAAG |
| | | GTCTCCAACAAAGCCCTCCCAGCCCCCATCGAGAAAACCAT |
| | | CTCCAAAGCCAAAGGGCAGCCCCGAGAACCACAGGTGTACA |
| | | CCCTGCCCCCATCCCGGGAGGAGATGACCAAGAACCAGGTC |
| | | AGCCTGACCTGCCTGGTCAAAGGCTTCTATCCCAGCGACAT |
| | | CGCCGTGGAGTGGGAGAGCAATGGGCAGCCGGAGAACAACT |
| | | ACAAGACCACGCCTCCCGTGCTGGACTCCGACGGCTCCTTC |
| | | TTCCTCTATAGCAAGCTCACCGTGGACAAGAGCAGGTGGCA |
| | | GCAGGGGAACGTCTTCTCATGCTCCGTGATGCATGAGGCTC |
| | | TGCACAACCACTACACGCAGAAGAGCCTCTCCCTGTCCCCG |
| | | GGTAAA |
| 51 | CL (kappa, Km3 | RTVAAPSVFIFPPSDEQLKSGTASVVCLLNNFYPREAKVQW |
| | allotype) | KVDNALQSGNSQESVTEQDSKDSTYSLSSTLILSKADYEKH |
| | | KVYACEVTHQGLSSPVIKSENRGEC |
| 52 | CL (kappa, Km3 | CGAACTGTGGCTGCACCATCTGTCTTCATCTTCCCGCCATC |
| | allotype) | TGATGAGCAGTTGAAATCTGGAACTGCCTCTGTTGTGTGCC |
| | | TGCTGAATAACTTCTATCCCAGAGAGGCCAAAGTACAGTGG |
| | | AAGGTGGATAACGCCCTCCAATCGGGTAACTCCCAGGAGAG |
| | | TGTCACAGAGCAGGACAGCAAGGACAGCACCIACAGCCTCA |
| | | GCAGCACCCTGACGCTGAGCAAAGCAGACTACGAGAAACAC |
| | | AAAGTCTACGCCTGCGAAGTCACCCATCAGGGCCTGAGCTC |
| | | GCCCGTCACAAAGAGCTTCAACAGGGGAGAGTGT |

### Example 3: Classical Humanisation of DOC2 and DOC3

Next, various humanized derivatives of DOC2 and DOC 3 were generated by applying commonly known strategies and procedures for antibody humanization. The following humanized variable heavy chains were generated.

TABLE 3

| SEQ ID No. | Description | Sequence |
|---|---|---|
| 21 | VH (DOC2RHA) | EVQLVESGGGVVQPGRSLRLSCAASGFTLSNYAMSWVRQAP |
| | | GKGLEWVAEVSSSGIYIYYSDTVTGRFTISRDNSKNTLYLQ |
| | | MNSLRAEDTAVYYCARDRYAYAMDYWGQGTLVTVSS |
| 2 | HCDR1 (DOC2RHA) | NYAMS |
| 3 | HCDR2 (DOC2RHA) | EVSSSGIYIYYSDTVTG |
| 4 | HCDR3 (DOC2RHA) | DRYAYAMDY |
| 22 | VH (DOC2RHA) | GAAGTGCAGCTGGTGGAATCCGGCGGAGGCGTGGTGCAGCC |
| | | TGGCAGATCCCTGAGACTGTCCTGCGCCGCCTCCGGCTTCA |
| | | CCCTGTCCAACTACGCCATGTCCTGGGTCCGACAGGCCCCT |
| | | GGCAAGGGCCTGGAATGGGTGGCCGAGGTGTCCTCCTCCGG |
| | | CATCTACATCTACTACTCCGACACCGTGACAGGCCGGTTCA |
| | | CCATCTCCCGGGACAACTCCAAGAACACCCTGTACCTGCAG |
| | | ATGAACTCCCTGCGGGCCGAGGACACCGCCGTGTACTACTG |
| | | CGCCAGAGACAGATACGCCTACGCCATGGACTACTGGGGCC |
| | | AGGGCACCCTGGTCACCGTGTCCTCT |
| 23 | VL (DOC2RKA) | DIVMTQSPDSLAVSLGERATINCKSSQSLLNSYNQKNSLAW |
| | | YQQKPGQPPKLLIYFASTRESGVPDRFSGSGSGTDFTLTIS |
| | | SLQAEDVAVYYCQQHYSNPRTFGGGTKLEIK |
| 7 | LCDR1 (DOC2RKA) | KSSQSLLNSYNQKNSLA |
| 8 | LCDR2 (DOC2RKA) | FASTRES |
| 9 | LCDR3 (DOC2RKA) | QQHYSNPRT |
| 24 | VL (DOC2RKA) | GACATCGTGATGACCCAGTCCCCCGACTCCCTGGCCGTGTC |
| | | TCTGGGCGAGCGGGCCACCATCAACTGCAAGTCCTCCCAGT |
| | | CCCTGCTGAACTCCTACAACCAGAAGAACAGCCTGGCGTGG |
| | | TATCAGCAGAAGCCCGGCCAGCCCCCCAAGCTGCTGATCTA |
| | | CTTCGCCTCCACCCGCGAGTCCGGCGTGCCCGATAGATTCT |
| | | CCGGCTCCGGCAGCGGCACCGACTTTACCCTGACCATCAGC |
| | | TCCCTGCAGGCCGAGGACGTGGCCGTGTACTACTGCCAGCA |
| | | GCACTACTCCAACCCCCGGACCTTCGGCGGAGGCACCAAGC |
| | | TGGAAATCAAA |
| 25 | VH (DOC3RH03) | EVQLVESGGGLVQPGGSLRLSCAVSGFSLTRYDLSWVRQAP |
| | | GKGLEWLGVIWTGGGTNYNSAFMSRLTISRDNSKNTLYLQM |
| | | NSLRAEDTAVYYCVRENYYNRRGYFDVWGQGTTVTVSS |
| 12 | HCDR1 (DOC3RH03) | RYDLS |
| 13 | HCDR2 (DOC3RH03) | VIWTGGGTNYNSAFMS |
| 14 | HCDR3 (DOC3RH03) | ENYYNRRGYFDV |
| 26 | VH (DOC3RH03) | GAAGTGCAGCTGGTCGAGTCTGGCGGAGGGCTCGTGCAGCC |
| | | TGGCGGATCTCTGAGACTGTCCTGTGCCGTGTCCGGCTTCA |
| | | GCCTGACCAGATACGACCTGTCCTGGGTCCGACAGGCTCCT |
| | | GGCAAGGGCCTGGAATGGCTGGGCGTGATCTGGACCGGCGG |
| | | AGGCACCAACTACAACTCCGCCTTCATGTCCCGGCTGACCA |
| | | TCTCCCGGGACAACTCCAAGAACACCCTGTACCTGCAGATG |
| | | AACTCCCTGCGGGCCGAGGACACCGCCGTGTACTACTGCGT |
| | | GCGCGAGAACTACTACAACAGACGGGGCTACTTCGACGTGT |
| | | GGGGCCAGGGCACCACTGTCACCGTGTCCTCC |
| 27 | VH (DOC3RHL) | QVQLQESDPGLVKPSETLSLTCTVSGFSISRYDLSWIRQPP |
| | | GKGLEWIGVIWTGGGTNYNSAFMSRLTISKDTSKNQVSLKL |
| | | SSVTAADTAVYYCARENYYNRRGYFDVWGQGTMVTVSS |
| 12 | HCDR1 (DOC3RHL) | RYDLS |
| 13 | HCDR2 (DOC3RHL) | VIWTGGGTNYNSAFMS |
| 14 | HCDR3 (DOC3RHL) | ENYYNRRGYFDV |
| 28 | VH (DOC3RHL) | GGCGAATTGGCGGAAGGCCGTCAAGGCCTAGGCGCGCCATG |
| | | AGCTCAAGCTTGCCGCCACCATGGAGTGGTCCGGCATCTTC |
| | | CTGTTTCTGGTGGCTACCGCCACCGACGTGCACTCTCAAGT |
| | | GCAGCTGCAAGAATCCGACCCCGGCCTCGTGAAACCCTCCG |
| | | AGACACTGTCCCTGACCTGCACCGTGTCCGGCTTCTCCATC |
| | | AGCAGATACGACCTGTCCTGGATCAGACAGCCCCCTGGCAA |
| | | GGGCCTGGAATGGATCGGCGTGATCTGGACCGGCGGAGGCA |
| | | CCAACTACAACTCCGCCTTCATGTCCCGGCTGACCATCTCC |
| | | AAGGACACCTCCAAGAACCAGGTGTCCCTGAAGCTGTCCTC |
| | | CGTGACCGCCGCTGACACCGCCGTGTACTACTGCGCCAGAG |
| | | AGAACTACTACAACAGACGGGGCTACTTCGACGTGTGGGGC |
| | | CAGGGCACCATGGTGACAGTGTCCTCCGCCTCCACCAAGGG |
| | | CCCGGTACCTCTTAATTAACTGGCCTCATGGGCCTTCCGCT |
| | | CACTGCCC |
| 29 | VL (DOC3RKA) | EIVLIQSPATLSLSPGERATLSCSASSSVSYMHWFQQKPGQ |
| | | APRLLIYLTSNLASGIPARFSGSGSGTDFTLTISSLEPEDF |
| | | AVYYCQQRSGSPPTFGPGTKLDIK |
| 17 | LCDR1 (DOC3RKA) | SASSSVSYMH |
| 18 | LCDR2 (DOC3RKA) | LTSNLAS |
| 19 | LCDR3 (DOC3RKA) | QQRSGSPPT |
| 30 | VL (DOC3RKA) | GAGATCGTGCTGACCCAGTCCCCTGCCACCCTGTCTCTGAG |
| | | CCCTGGCGAGAGAGCCACCCTGAGCTGCTCCGCCTCCTCCT |
| | | CCGTGTCCTACATGCACTGGTTCCAGCAGAAGCCCGGCCAG |
| | | GCCCCTCGGCTGCTGATCTACCTGACCTCCAACCTGGCCTC |
| | | CGGCATCCCTGCCCGGTTCTCTGGCTCCGGCTCCGGCACCG |
| | | ACTTTACCCTGACCATCTCCAGCCTGGAACCCGAGGACTTC |
| | | GCCGTGTACTACTGCCAGCAGCGGAGCGGCTCCCCCCCTAC |
| | | ATTTGGCCCTGGCACCAAGCTGGACATCAAA |

Altogether three antibodies were produced utilizing aforementioned humanized variable heavy and variable light chains:
Antibody DOC2-reshaped contains fully human constant domains, the variable heavy chain of DOC2RHA (SEQ ID NO. 21) and the variable light chain of DOC2RKA (SEQ ID NO. 23),
Antibody DOC3-RH03XRKA contains fully human constant domains, the variable heavy chain of DOC3RH03 (SEQ ID NO. 25) and the variable light chain of DOC3RKA (SEQ ID No. 29), and
Antibody DOC3-RHLXRKA contains fully human constant domains, the variable heavy chain of DOC3RHL (SEQ ID NO. 27) and the variable light chain of DOC3RKA (SEQ ID No. 29).

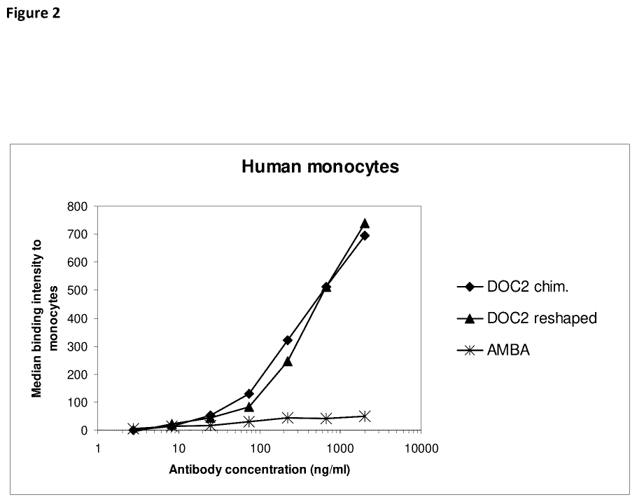

The binding of DOC2-reshaped to human PBMCs was compared to DOC2-chim. The antibody AMBA served as negative control. Results are shown in FIG. 2. The binding of DOC2-reshaped was comparable and only slightly inferior compared to DOC2-chim.

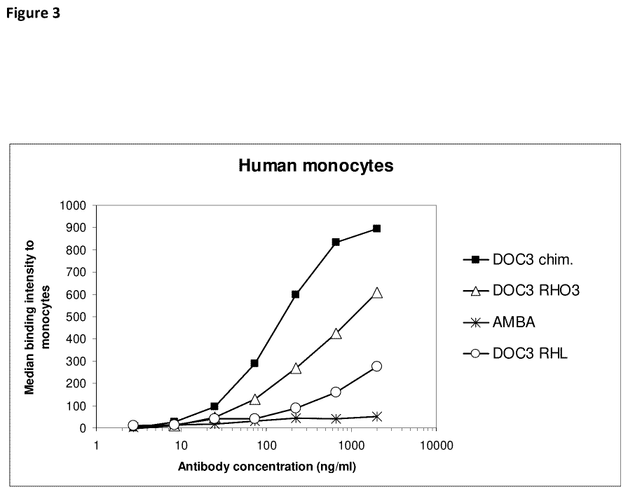

The same experiment was performed with antibodies DOC3-RH03XRKA and DOC3-RHLXRKA, in comparison to DOC3-chim. Results are shown in FIG. 3. DOC3 RH03XRKA and DOC3 RHLXRKA showed a reduced binding compared to DOC3 chim. antibody, indicating that some binding activity was lost during humanization.

In the next experiment, the humanized binders were tested for crossreactivity with marmoset CCR2 using marmoset blood.

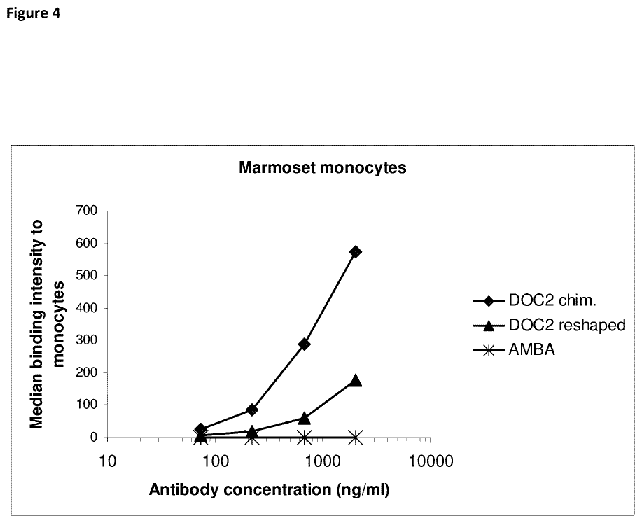

Results for the humanized DOC2 antibody are shown in FIG. 4. The binding activity of antibody DOC2-reshaped to primary marmoset monocytes was markedly reduced compared to the DOC2 chim. This result was surprising since DOC2-reshaped showed the same binding activity primary human monocytes as DOC2-chim.

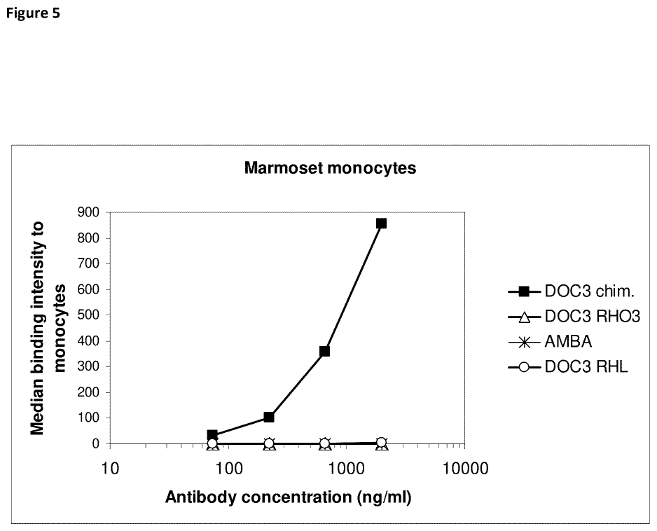

Results for the humanized DOC3 antibodies are shown in FIG. 5. Results were even more drastic. DOC3-RH03XRKA and DOC3-RHLXRKA showed a complete loss of binding to primary marmoset monocytes.

Taken together these data show that a common strategies for humanization are not suitable to obtain humanized antibodies that bind to human CCR2 and still cross-react with marmoset CCR2. Cross-reactivity with marmoset CCR2 is considered to be essential for preclinical development of the antibodies, for example to collect safety and other preclinical data in non-human primates.

### Example 4: Additional Attempts for Humanisation of of DOC2 and DOC3

Next, following additional humanized derivatives of DOC2 and DOC 3 were generated. The following humanized variable chains were generated.

TABLE 4

| SEQ ID No. | Description | Sequence |
|---|---|---|
| 31 | VL (DOC2RKB) | DIVMTQSPDSLAVSLGERATINCKSSQSLLNSYNQKNSLAW |
| | | YQQKPGQPPKLLVYFASTRESGVPDRFSGSGSGTDFTLTIS |
| | | SLQAEDVAVYFCQQHYSNPRTFGGGTKLEIK |
| 7 | LCDR1 (DOC2RKB) | KSSQSLLNSYNQKNSLA |
| 8 | LCDR2 (DOC2RKB) | FASTRES |
| 9 | LCDR3 (DOC2RKB) | QQHYSNPRT |
| 32 | VL (DOC2RKB) | GACATCGTGATGACCCAGTCCCCCGACTCCCTGGCCGTGTC |
| | | TCTGGGCGAGCGGGCCACCATCAACTGCAAGTCCTCCCAGT |
| | | CCCTGCTGAACTCCTACAACCAGAAGAACAGCCTGGCGTGG |
| | | TATCAGCAGAAGCCCGGCCAGCCCCCCAAGCTGCTGGTGTA |
| | | CTTCGCCTCCACCCGCGAGTCTGGCGTGCCCGACAGATTCT |
| | | CCGGCTCCGGCAGCGGCACCGACTTTACCCTGACCATCAGC |
| | | TCCCTGCAGGCCGAGGACGTGGCCGTGTACTTCTGCCAGCA |
| | | GCACTACTCCAACCCCCGGACCTTCGGCGGAGGCACCAAGC |
| | | TGGAAATCAAA |
| 33 | VH (DOC2RHB) | EVOLVESGGGVVQPGRSLRLSCAASGFTLSNYAMSWVRQAP |
| | | GKGLEWVAEVSSSGIYIYYSDTVTGRFSISRDNSKNTLYLQ |
| | | MNSLRAEDTAVYYCARDRYAYAMDYWGQGTLVIVSS |
| 2 | HCDR1 (DOC2RHB) | NYAMS |
| 3 | HCDR2 (DOC2RHB) | EVSSSGIYIYYSDTVTG |
| 4 | HCDR3 (DOC2RHB) | DRYAYAMDY |
| 34 | VH (DOC2RHB) | GAAGTGCAGCTGGTGGAATCCGGCGGAGGCGTGGTGCAGCC |
| | | TGGCAGATCCCTGAGACTGTCCTGCGCCGCCTCCGGCTTCA |
| | | CCCTGTCCAACTACGCCATGTCCTGGGTGCGACAGGCCCCT |
| | | GGCAAGGGACTGGAATGGGTGGCCGAGGTGTCCTCCTCCGG |
| | | CATCTACATCTACTACTCCGACACCGTGACCGGCCGGTTCT |
| | | CCATCTCCCGGGACAACTCCAAGAACACCCTGTACCTGCAG |
| | | ATGAATTCCCTGCGGGCCGAGGACACCGCCGTGTACTACTG |
| | | CGCCAGAGACAGATACGCCTACGCCATGGACTACTGGGGCC |
| | | AGGGCACCCTGGTGATCGTGTCCTCC |
| 35 | VL (DOC2RKC) | DIVMTQSPDSLAVSLGERATINCKSSQSLLNSYNQKNSLAW |
| | | YQQKPGQPPKLLVYFASTRESGVPDRFIGSGSGTYFTLTIS |
| | | SLQAEDVAVYFCQQHYSNPRTFGGGTKLEIK |
| 7 | LCDR1 (DOC2RKC) | KSSQSLLNSYNQKNSLA |
| 8 | LCDR2 (DOC2RKC) | FASTRES |
| 9 | LCDR3 (DOC2RKC) | QQHYSNPRT |
| 36 | VL (DOC2RKC) | GACATCGTGATGACCCAGTCCCCCGACTCCCTGGCCGTGTC |
| | | TCTGGGCGAGCGGGCCACCATCAACTGCAAGTCCTCCCAGT |
| | | CCCTGCTGAACTCCTACAACCAGAAGAACAGCCTGGCCTGG |
| | | TATCAACAGAAGCCCGGCCAGCCCCCCAAGCTGCTGGTGTA |
| | | CTTCGCCTCCACCCGCGAGTCCGGCGTGCCCGACAGATTCA |
| | | TCGGCTCCGGCTCCGGCACCIACTTCACCCTGACCATCAGC |
| | | TCCCTGCAGGCCGAGGACGTGGCCGTGTACTTCTGCCAGCA |
| | | GCACTACTCCAACCCCCGGACCTTCGGCGGAGGCACCAAGC |
| | | TGGAAATCAAA |
| 37 | VH (DOC3RH09) | EVOLVESGGGLVQPGGSLRLSCAVSGFSLTRYDLSWVRQAP |
| | | GKGLEWLGVIWTGGGTNYNSAFMSRFSISKDNSKNTVFLQM |
| | | NSLRAEDTAVYYCARENYYNRRGYFDVWGQGTTVTVSS |
| 12 | HCDR1 (DOC3RH09) | RYDLS |
| 13 | HCDR2 (DOC3RH09) | VIWTGGGTNYNSAFMS |
| 14 | HCDR3 (DOC3RH09) | ENYYNRRGYFDV |
| 38 | VH (DOC3RH09) | GAAGTGCAGCTGGTGGAATCCGGCGGAGGACTGGTGCAACC |
| | | TGGCGGCTCCCTGAGACTGTCTTGCGCCGTGTCCGGCTTCA |
| | | GCCTGACCAGATACGACCTGTCCTGGGTGCGACAGGCCCCT |
| | | GGCAAGGGCCTGGAATGGCTGGGCGTGATCTGGACCGGAGG |
| | | AGGCACCAACTACAACTCCGCCTTCATGTCCCGGTTCTCCA |
| | | TCTCCAAGGACAACTCCAAGAACACCGTGTTTCTGCAGATG |
| | | AACTCCCTGCGGGCTGAGGACACCGCCGTGTACTACTGCGC |
| | | CAGAGAGAACTACTACAACAGACGGGGCTACTTCGACGTGT |
| | | GGGGCCAGGGCACCACCGTGACCGTGTCCTCT |
| 39 | VL (DOC3RKC) | QIVLTQSPATLSLSPGERATLSCSASSSVSYMHWFQQKPGQ |
| | | APRLLIYLTSNLASGVPARFSGSGSGTDFTLTISSLEPEDF |
| | | AVYYCQQRSGSPPTFGPGTKLDIK |
| 17 | LCDR1 (DOC3RKC) | SASSSVSYMH |
| 18 | LCDR2 (DOC3RKC) | LTSNLAS |
| 19 | LCDR3 (DOC3RKC) | QQRSGSPPT |
| 40 | VL (DOC3RKC) | CAGATCGTGCTGACCCAGTCCCCTGCCACCCTGTCTCTGAG |
| | | CCCTGGCGAGAGAGCCACCCTGAGCTGCTCCGCCTCCTCCT |
| | | CCGTGTCCTACATGCACTGGTTCCAGCAGAAGCCCGGCCAG |
| | | GCCCCTCGGCTGCTGATCTACCTGACCTCCAACCTGGCCTC |
| | | CGGCGTGCCCGCCAGATTCTCTGGCTCCGGCTCCGGCACCG |
| | | ACTTCACCCTGACCATCTCCAGCCTGGAACCCGAGGACTTC |
| | | GCCGTGTACTACTGCCAGCAGCGGAGCGGCTCCCCCCCTAC |
| | | CTTTGGCCCTGGCACCAAGCTGGACATCAAA |

The following antibodies were tested in subsequent experiments:
Antibody DOC2-RHAXRKA aka DOC2-reshaped containing fully human constant domains, the variable heavy chain of DOC2RHA (SEQ ID NO. 21) and the variable light chain of DOC2RKA (SEQ ID NO. 23),
Antibody DOC2-RHAXRKB containing fully human constant domains, the variable heavy chain of DOC2RHA (SEQ ID NO. 21) and the variable light chain of DOC2RKB (SEQ ID No. 31),
Antibody DOC2-RHBXRKA containing fully human constant domains, the variable heavy chain of DOC2RHB (SEQ ID NO. 33) and the variable light chain of DOC2RKA (SEQ ID No. 23),
Antibody DOC2-RHBXRKB containing fully human constant domains, the variable heavy chain of DOC2RHB (SEQ ID NO. 33) and the variable light chain of DOC2RKA (SEQ ID No. 31),
Antibody DOC2-RHBXRKC containing fully human constant domains, the variable heavy chain of DOC2RHB (SEQ ID NO. 33) and the variable light chain of DOC2RKC (SEQ ID No. 35),
Antibody DOC2-ChXRKA containing fully human constant domains, a chimeric variable heavy chain and the variable light chain of DOC2RKA (SEQ ID No. 23),
Antibody DOC-2 chim.,
Antibody DOC3-RH09XRKA containing fully human constant domains, the variable heavy chain of DOC3RH09 (SEQ ID NO. 37) and the variable light chain of DOC3RKA (SEQ ID No. 29),
Antibody DOC3-RH09XRKC containing fully human constant domains, the variable heavy chain of DOC3RH09 (SEQ ID NO. 37) and the variable light chain of DOC3RKC (SEQ ID No. 39), and
Antibody DOC3-CHXRKA containing fully human constant domains, a chimeric variable heavy chain and the variable light chain of DOC3RKA (SEQ ID No. 29),

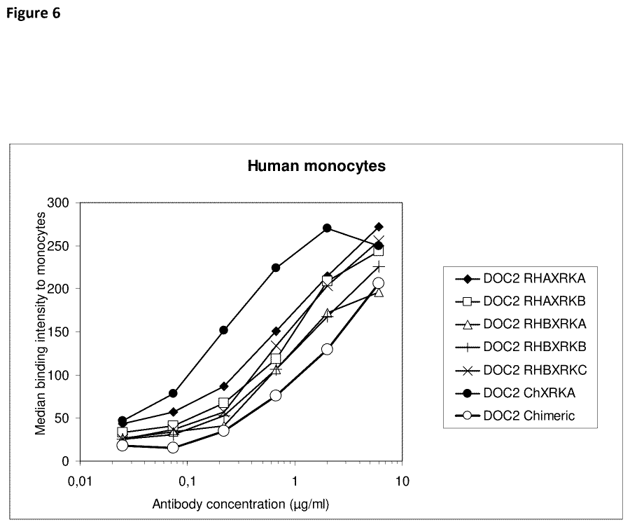

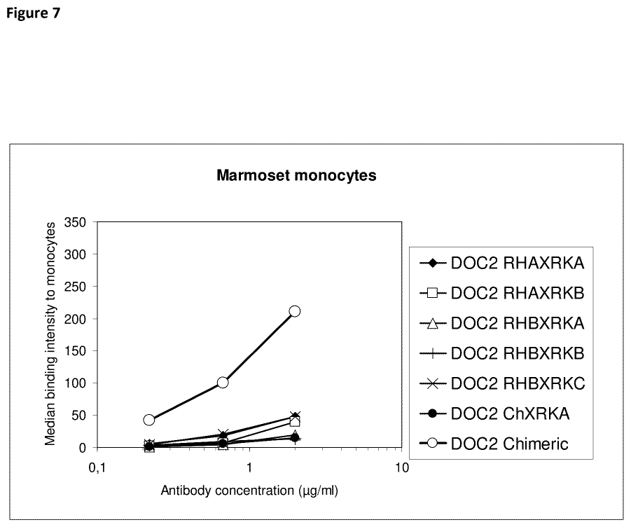

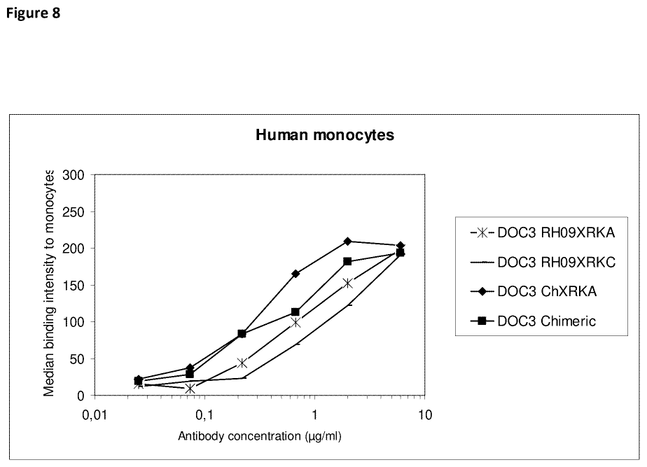

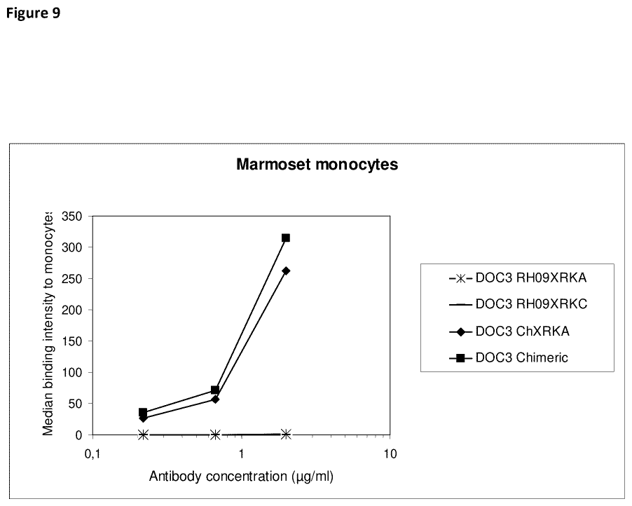

In the next experiment, the additionally humanized DOC2 and DOC3 binders were tested for binding to primary human monocytes and primary marmoset monocytes. Binding of the humanized DOC2 binders to human monocytes are shown in FIG. 6. Binding of the humanized DOC2 binders to marmoset monocytes are shown in FIG. 7. Binding of the humanized DOC3 binders to human monocytes are shown in FIG. 8. Binding of the humanized DOC3 binders to marmoset monocytes are shown in FIG. 9.

For the DOC2 derivatives, again all humanized variants, including the variant with only a humanized light chain (DOC2 CHXRKA), did show good binding to primary human monocytes, but not to primary marmoset monocytes. These results suggest that the light chain of DOC2 is critical for cross-reactivity with primary marmoset monocytes.

For the DOC3 derivatives, all humanized variants (Doc3RH09XRKA, Doc3RH09XRKC) did show good binding to primary human monocytes, but not to primary marmoset monocytes. A variant with only a humanized light chain (DOC3 CHXRKA) shows full cross-reactivity with primary marmoset monocytes. These data suggest that the heavy chain of DOC3 is critical for cross-reactivity with primary marmoset monocytes.

Taken together, the results show that cross-reactivity of DOC2 and DOC3 to primary marmoset monocytes is extremely difficult to retain during the humanization procedure.

### Example 5: Further Humanisation of of DOC2 with Additional Variable Light Chains

Since the light chain of DOC2 seems to be critical for cross-reactivity with primary marmoset monocytes, several additional humanized variants of light chain of DOC2 were generated and tested together with the DOC2RHA heavy chain for binding to marmoset monocytes. The following additional humanized variable light chains were generated with the rational to go back to the original murine frameworks.

TABLE 5

| SEQ ID No. | Description | Sequence |
|---|---|---|
| 41 | VL (DOC2RKA FR1) | DIVMTQSPSSLAISVGQRVTLSCKSSQSLLNSYNQKNSLAW |
| | | YQQKPGQPPKLLIYFASTRESGVPDRFSGSGSGTDFTLTIS |
| | | SLQAEDVAVYYCQQHYSNPRTFGGGTKLEIK |
| 7 | LCDR1 (DOC2RKA FR1) | KSSQSLLNSYNQKNSLA |
| 8 | LCDR2 (DOC2RKA FR1) | FASTRES |
| 9 | LCDR3 (DOC2RKA FR1) | QQHYSNPRT |
| 42 | VL (DOC2RKA FR1) | GACATCGTGATGACCCAGTCCCCCTCCAGCCTGGCCATCTC |
| | | CGTGGGCCAGAGAGTGACCCTGTCCTGCAAGTCCTCCCAGT |
| | | CCCTGCTGAACTCCTACAACCAGAAGAACAGCCTGGCGTGG |
| | | TATCAGCAGAAGCCCGGCCAGCCCCCCAAGCTGCTGATCTA |
| | | CTTCGCCTCCACCCGCGAGTCCGGCGTGCCCGATAGATTCT |
| | | CCGGCTCCGGCAGCGGCACCGACTTTACCCTGACCATCAGC |
| | | TCCCTGCAGGCCGAGGACGTGGCCGTGTACTACTGCCAGCA |
| | | GCACTACTCCAACCCCCGGACCTTCGGCGGAGGCACCAAGC |
| | | TGGAAATCAAA |
| 43 | VL (DOC2RKA FR2) | DIVMTQSPDSLAVSLGERATINCKSSQSLLNSYNQKNSLAW |
| | | YQQKPGQSPKLLVYFASTRESGVPDRFSGSGSGTDFTLTIS |
| | | SLQAEDVAVYYCQQHYSNPRTFGGGTKLEIK |
| 7 | LCDR1 (DOC2RKA FR2) | KSSQSLLNSYNQKNSLA |
| 8 | LCDR2 (DOC2RKA FR2) | FASTRES |
| 9 | LCDR3 (DOC2RKA FR2) | QQHYSNPRT |
| 44 | VL (DOCZRKA FR2) | GACATCGTGATGACCCAGTCCCCCGACTCCCTGGCCGTGTC |
| | | TCTGGGCGAGCGGGCCACCATCAACTGCAAGTCCTCCCAGT |
| | | CCCTGCTGAACTCCTACAACCAGAAGAACAGCCTGGCGTGG |
| | | TATCAGCAGAAGCCCGGCCAGTCCCCCAAGCTGCTGGTGTA |
| | | CTTCGCCTCCACCCGCGAGTCCGGCGTGCCCGATAGATTCT |
| | | CCGGCTCCGGCAGCGGCACCGACTTTACCCTGACCATCAGC |
| | | TCCCTGCAGGCCGAGGACGTGGCCGTGTACTACTGCCAGCA |
| | | GCACTACTCCAACCCCCGGACCTTCGGCGGAGGCACCAAGC |
| | | TGGAAATCAAA |
| 45 | VL (DOC2RKA FR3a) | DIVMTQSPDSLAVSLGERATINCKSSQSLLNSYNQKNSLAW |
| | | YQQKPGQPPKLLIYFASTRESGVPDRFIGSGSGTYFTLTIT |
| | | SLQAEDVAVYYCQQHYSNPRTFGGGTKLEIK |
| 7 | LCDR1 (DOC2RKA | KSSQSLLNSYNQKNSLA |
| | FR3a) | |
| 8 | LCDR2 (DOC2RKA | FASTRES |
| | FR3a) | |
| 9 | LCDR3 (DOC2RKA | QQHYSNPRT |
| | FR3a) | |
| 46 | VL (DOC2RKA FR3a) | GACATCGTGATGACCCAGTCCCCCGACTCCCTGGCCGTGTC |
| | | TCTGGGCGAGCGGGCCACCATCAACTGCAAGTCCTCCCAGT |
| | | CCCTGCTGAACTCCTACAACCAGAAGAACAGCCTGGCGTGG |
| | | TATCAGCAGAAGCCCGGCCAGCCCCCCAAGCTGCTGATCTA |
| | | CTTCGCCTCCACCCGCGAGTCCGGCGTGCCCGACAGATTCA |
| | | TCGGCTCCGGCTCCGGCACCIACTTCACCCTGACCATCACC |
| | | TCCCTGCAGGCCGAGGACGTGGCCGTGTACTACTGCCAGCA |
| | | GCACTACTCCAACCCCCGGACCTTCGGCGGAGGCACCAAGC |
| | | TGGAAATCAAA |
| 47 | VL (DOC2RKA FR3b) | DIVMTQSPDSLAVSLGERATINCKSSQSLLNSYNQKNSLAW |
| | | YQQKPGQPPKLLIYFASTRESGVPDRFSGSGSGTDFTLTIS |
| | | SVQAGDLADYFCQQHYSNPRTFGGGTKLEIK |
| 7 | LCDR1 (DOC2RKA | KSSQSLLNSYNQKNSLA |
| | FR3b) | |
| | LCDR2 (DOC2RKA | FASTRES |
| | FR3b) | |
| 9 | LCDR3 (DOC2RKA | QQHYSNPRI |
| | FR3b) | |
| 48 | VL (DOC2RKA FR3b) | GACATCGTGATGACCCAGTCCCCCGACTCCCTGGCCGTGTC |
| | | TCTGGGCGAGCGGGCCACCATCAACTGCAAGTCCTCCCAGT |
| | | CCCTGCTGAACTCCTACAACCAGAAGAACAGCCTGGCGTGG |
| | | TATCAGCAGAAGCCCGGCCAGCCCCCCAAGCTGCTGATCTA |
| | | CTTCGCCTCCACCCGCGAGTCCGGCGTGCCCGATAGATTCT |
| | | CCGGCTCCGGCAGCGGCACCGACTTTACCCTGACCATCAGC |
| | | TCCGTGCAGGCCGGCGACCTGGCCGACTACTTCTGCCAGCA |
| | | GCACTACTCCAACCCCCGGACCTTCGGCGGAGGCACCAAGC |
| | | TGGAAATCAAA |

The following antibodies were tested in subsequent experiments:
Antibody DOC2-RHAXRKA FR1 containing fully human constant domains, the variable heavy chain of DOC2RHA (SEQ ID NO. 21) and the variable light chain of DOC2RKA FR1 (SEQ ID NO. 41),
Antibody DOC2-RHAXRKA FR2 containing fully human constant domains, the variable heavy chain of DOC2RHA (SEQ ID NO. 21) and the variable light chain of DOC2RKA FR2 (SEQ ID NO. 43),
Antibody DOC2-RHAXRKA FR3a containing fully human constant domains, the variable heavy chain of DOC2RHA (SEQ ID NO. 21) and the variable light chain of DOC2RKA FR3a (SEQ ID NO. 45), and
Antibody DOC2-RHAXRKA FR3b containing fully human constant domains, the variable heavy chain of DOC2RHA (SEQ ID NO. 21) and the variable light chain of DOC2RKA FR3b (SEQ ID NO. 47).

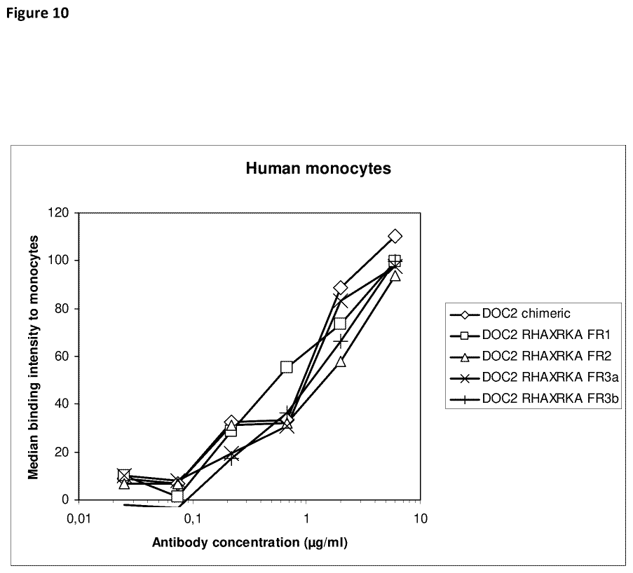

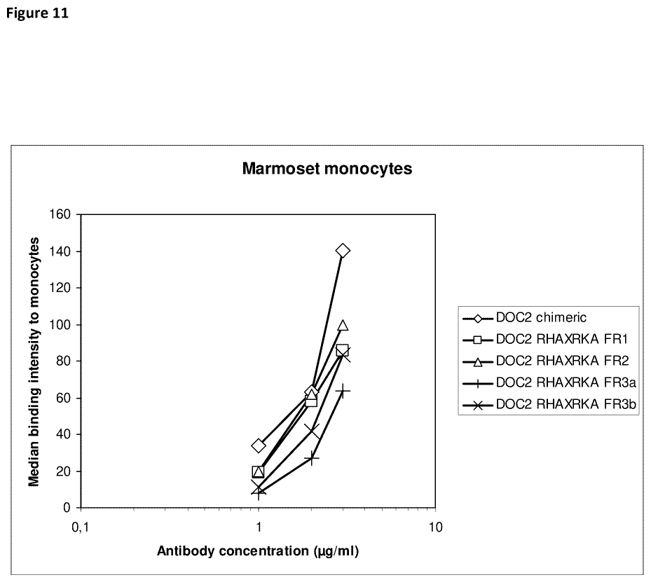

Again, antibodies were tested for binding to primary human monocytes and primary marmoset monocytes. Binding to human monocytes is shown in FIG. 10. Binding to marmoset monocytes is shown in FIG. 11. The data show that DOC2 RHAXRKA FR2 has an acceptable crossreactivity with primary marmoset monocytes. This variant was therefore selected for depletion of monocytes in marmosets, as well as for pharmacokinetic and toxicological studies in marmosets.

### Example 6: Summary of the Humanisation Procedures

Standard approaches for humanization of the anti-CCR2 antibodies DOC2 and DOC3 were not successful. Humanized variants showed good binding to human-CCR2 transfected CHO cells and primary human monocytes, but almost completely lost their cross-reactivity to primary marmoset monocytes. Cross-reactivity with marmoset monocytes is an essential property, since antibody mediated depletion of monocytes has not been performed in humans or non-human primates, and preclinical data about safety of monocyte depletion in non-human primates are essential for clinical development. In addition, it was so far not shown in non-human primates, that depletion of monocytes is possible with a monoclonal antibody directed against CCR2.

Multiple variants had to be generated by trial and error, since the classical strategy for humanization of DOC-2 and DOC-3 resulted in antibodies that did not cross-react with marmoset CCR2. Finally a humanized DOC2 antibody could be generated that retained cross-reactivity to marmoset CCR2. This humanized DOC2 antibody, DOC2 RHAXRKA FR2, retains the light chain framework regions required for marmoset cross-reactivity. No humanized DOC3 antibody could be identified that retained cross-reactivity to marmoset CCR2.

Antibody DOC-2 recognizes a three-dimensional epitope consisting of the first extracellular loop and the first half of the second extracellular loop of CCR2. DOC-2 cross-reacts with marmoset CCR2, but not with rhesus CCR2. Sequence comparison of human, marmoset and rhesus CCR2 reveals that the first extracellular loop is identical in all three species. In the second extracellular loop rhesus CCR2 has two amino acid exchanges (Lys→Glu and Val→Ile), while marmoset has only one conservative exchange (Cys→Ser).

TABLE 6

| | First | Second |
|---|---|---|
| | extra- | extra- |
| | cellular | cellular |
| | loop | loop |
| Human | WVFG | FTKCQKEDSV |
| CCR2 | (SEQ ID | YVCGPYFPRGW |
| | No. 53) | (SEQ ID No. 54) |
| Marmoset | WVFG | FTKSQKEDSVY |
| CCR2 | (SEQ ID | VCGPYFPRGW |
| | No. 53) | (SEQ ID No. 55) |
| Rhesus | WVFG | FTKCQEEDSV |
| CCR2 | (SEQ ID | YICGPYFPRGW |
| | No. 53) | (SEQ ID No. 56) |

The Cys to Ser exchange in marmoset CCR2 may be the reason, why subtle changes in the binding mode of the humanized DOC-2 antibodies resulted in a loss of cross-reactivity to marmoset CCR2, which is considered an essential property for preclinical safety studies of a cell depleting antibody.

### Example 7: ADCC Activity of Humanized DOC2 Antibody

In this experiment the activity of humanized antibody DOC2-RHAXRKA FR2 was compared to rituximab and a human IgG1 isotype control antibody.

PBMC's were prepared by Ficoll-Paque density gradient centrifugation. As culture medium RPMI1640+10% FCS+1% PenStrep+1% L-glutamine was used. 0.5 million of freshly prepared PBMC's were grown in 200 μl culture medium 96-well plates (U-bottom). Antibodies were added to the wells in serial dilutions. Cells were incubated for 24 hours at 37° C. and then stained with the following directly labeled antibodies: anti-CD16-APC, anti-CD11b-PECy7, anti-CD3-APCCy7, anti-CD14-AmCyan, anti-CD19-PacificBlue for 30 min on ice. Cells were washed with PBS and counting beads (10.000/well) were added to quantify absolute cell numbers/well Cells were analyzed with a BD CANTO-II FACS machine and FACS-DIVA analysis software.

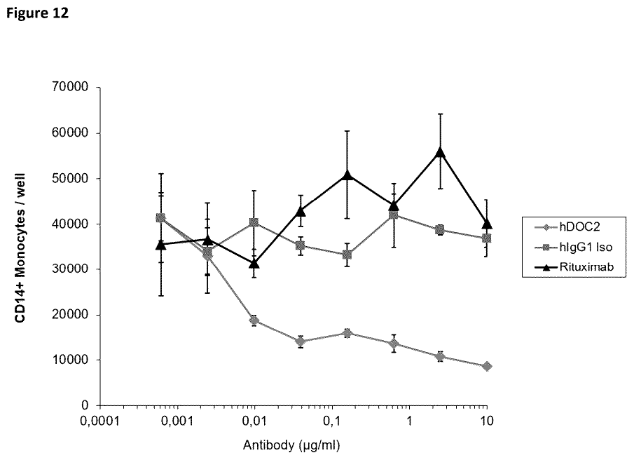

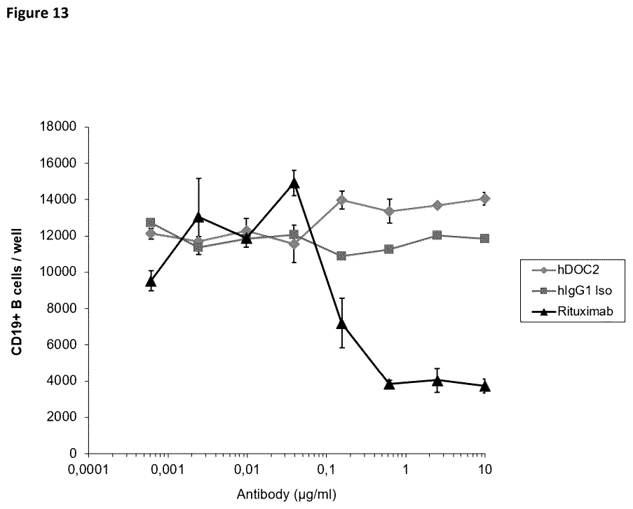

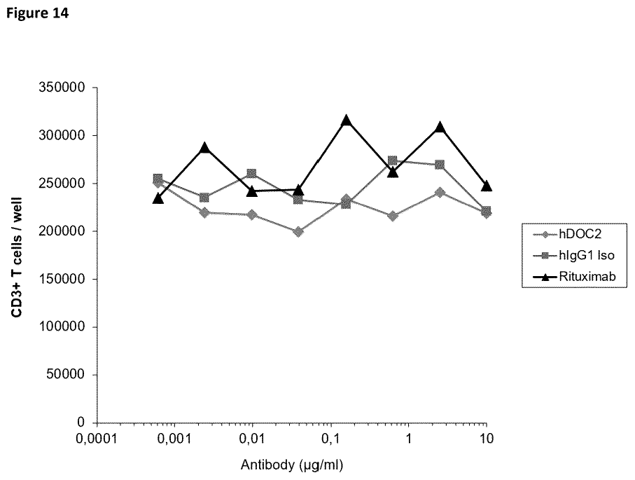

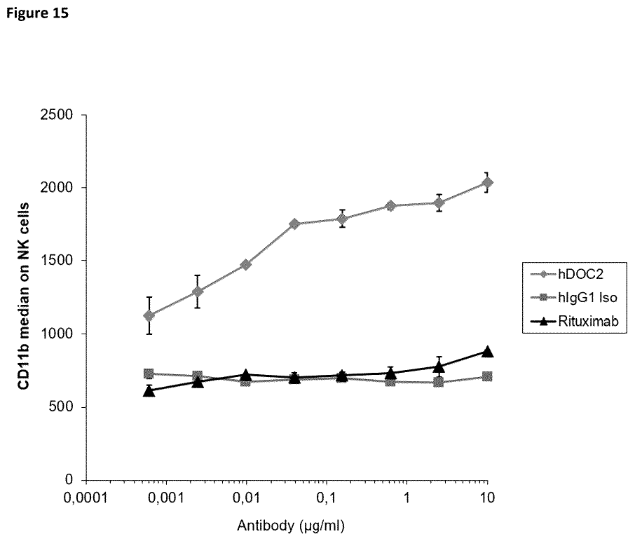

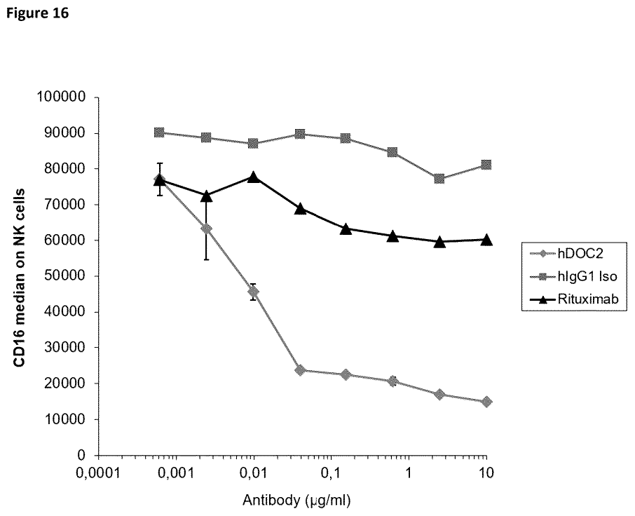

Results are shown in FIG. 12-16. DOC2-RHAXRKA FR2 (hDOC2) significantly depleted classical CD14+ monocytes (which express high levels of CCR2; FIG. 12), but not B cells (FIG. 13) or T cells (FIG. 14). DOC2-RHAXRKA FR2 also strongly activates NK cells, as indicated by the upregulation of CD11b (FIG. 15) and the downregulation of CD16 (FIG. 16) on NK cells. Rituximab depletes the B cells, but not other cell types.

1.-12. (canceled)

13. A humanized antibody or antibody fragment that specifically binds to human C-C Motif Chemokine Receptor 2 (CCR2) comprising a variable heavy chain (VH) comprising HCDR1, HCDR2, and HCDR3, and a variable light chain (VL) comprising LCDR1, LCDR2, and LCDR3, wherein the HCDR1, HCDR2, and HCDR3 comprise the HCDR1, HCDR2, and HCDR3 amino acid sequences of the VH amino acid sequence set forth in SEQ ID NO: 21; and wherein the LCDR1, LCDR2, and LCDR3 comprise the LCDR1, LCDR2, and LCDR3 of the VL amino acid sequence set forth in SEQ ID NO: 43.

14. The humanized antibody or antibody fragment of claim 13, wherein the HCDR1, HCDR2, HCDR3, LCDR1, LCDR2, and LCDR2 comprise the amino acid sequences set forth is SEQ ID NO: 10, SEQ ID NOs: 2, 3, 4, 7, 8, and 9, respectively.

15. The humanized antibody or antibody fragment of claim 13, wherein the VH comprises the amino acid sequence set forth in SEQ ID NO: 21.

16. The humanized antibody or antibody fragment of claim 13, wherein the VL comprises the amino acid sequence set forth in SEQ ID NO: 43.

17. The humanized antibody or antibody fragment of claim 13, which is an antibody of the human IgG1 class.

18. The humanized antibody or antibody fragment of claim 13, wherein the antibody or antibody fragment is cross-reactive with marmoset CCR2, but not rhesus CCR2.

19. A humanized antibody or antibody fragment that specifically binds to CCR2 comprising a VH comprising the amino acid sequence set forth in SEQ ID NO: 21, and a VL comprising the amino acid sequence set forth in SEQ ID NO: 43.

20. The humanized antibody or antibody fragment of claim 19, which is an antibody of the human IgG1 class.

21. The humanized antibody or antibody fragment of claim 19, wherein the antibody or antibody fragment is cross-reactive with marmoset CCR2, but not rhesus CCR2.

22. A nucleic acid sequence or a plurality of nucleic acid sequences encoding a VH and/or a VL, or a heavy chain and/or a light chain of the humanized antibody of claim 13.

23. A vector or plurality of vectors comprising the nucleic acid sequence or plurality of nucleic acid sequences of claim 22.

24. A host cell comprising the nucleic acid sequence or plurality of nucleic acid sequences of claim 22.

25. A method for producing an antibody that binds to CCR2, comprising culturing the host cell of claim 24 under conditions which permit expression of the antibody.

26. A composition comprising the humanized antibody or antibody fragment of claim 13 and at least one pharmaceutically acceptable carrier.

27. A nucleic acid sequence or a plurality of nucleic acid sequences encoding a VH and/or a VL, or a heavy chain and/or a light chain of the humanized antibody of claim 19.

28. A vector or plurality of vectors comprising the nucleic acid sequence or plurality of nucleic acid sequences of claim 27.

29. A host cell comprising the nucleic acid sequence or plurality of nucleic acid sequences of claim 27.

30. A composition comprising the humanized antibody or antibody fragment of claim 19 and at least one pharmaceutically acceptable carrier.

31. A method of treating an inflammatory disease or an autoimmune disease in a subject in need thereof comprising administering a therapeutically effective amount of the humanized antibody or antibody fragment of claim 13 to the subject.

32. A method of treating a proliferative disease in a subject in need thereof comprising administering a therapeutically effective amount of the humanized antibody or antibody fragment of claim 13 to the subject.
